(19) United States
(12) Patent Application Publication
(10) Pub. No.: US 2026/0283635 A1
(43) Pub. Date: Sep. 24, 2026

(54) Balloon Dilation Devices, Methods of Use, and Methods of Manufacture

(71) Applicant: ENTELLUS MEDICAL, INC., Plymouth, MN (US)

(72) Inventors: Jarrod Neuharth, Coon Rapids, MN (US); Ryan Cerepak, Champlin, MN (US); John Drontle, Buffalo, MN (US); Aaron Pidde, Bloomington, MN (US); Alexander Houck, Minnetonka, MN (US); Paul Lesch, Lino Lakes, MN (US); Anthony J. Hanson, Chaska, MN (US); Daniel Flo, Shorewood, MN (US)

(21) Appl. No.: 19/471,622

(22) PCT Filed: Apr. 3, 2024
(86) PCT No.: PCT/US2024/022900

§ 371 Date: Oct. 1, 2025

Related U.S. Application Data
(60) Provisional application No. 63/457,127, filed on Apr. 4, 2023.

(51) Int. Cl. A61B 17/24; A61B 17/00; A61B 34/20; A61M 25/01; A61M 29/02

(52) U.S. Cl. CPC A61B 17/24; A61B 34/20; A61M 25/0105; A61M 29/02; A61B 2017/00526; A61B 2017/246; A61B 2034/2051; A61M 2025/0166

(57) ABSTRACT

In an example, a balloon dilation device includes a handle having a proximal handle end and a distal handle end. The balloon dilation device also includes a shaft extending distally from the distal handle end, and an inflatable balloon disposed about at least a portion of a distal portion of the shaft. The shaft includes a hypotube having an inner surface and an outer surface. The inner surface defines a lumen of the hypotube. The shaft also includes a liner tube disposed within the lumen of the hypotube, and a position sensor coupled to a distal portion of the hypotube. The position sensor is between the inner surface of the hypotube and the liner tube.

### CROSS-REFERENCE TO RELATED APPLICATION

The present application claims the benefit of priority to U.S. Provisional Application No. 63/457,127, filed Apr. 4, 2023, the contents of which is hereby incorporated by reference in its entirety.

### FIELD

The present disclosure generally relates to balloon dilation devices and, in particular, a balloon dilation device including a sensor for image guided surgery.

### BACKGROUND

Balloon dilation devices may be used during a sinuplasty procedure and/or a Eustachian tube procedure to treat chronic sinusitis and/or Eustachian tube dysfunction (ETD), respectively. In general, these procedures involve inserting a balloon dilation device into a nasal cavity and navigating the balloon dilation device through the nasal cavity to an outflow tract of an affected sinus cavity or the Eustachian tube. A balloon at a distal end of the balloon dilation device is then inflated under controlled pressure, effectively dilating the narrow or obstructed sinus passages or Eustachian tube to restore normal function (e.g., sinus drainage or flow of mucus and air through the Eustachian tube).

To aid navigating the balloon dilation device through the nasal cavity, a sensor may be attached to the balloon dilation device to provide for image guided surgery. Image guided surgery allows for real-time visualization of a position of the balloon dilation device relative to images of the nasal cavity (e.g., including the sinus cavities and/or the Eustachian tube) and helps to ensure precise and accurate placement of the balloon dilation device. This can be beneficial in the treatment of chronic sinusitis, where the sinus passages are often narrow and difficult to access. Image guided sinuplasty provides the surgeon with information to perform the procedure with increased precision, resulting in improved patient outcomes and reduced risk of complications.

### BRIEF DESCRIPTION OF THE FIGURES

The novel features believed characteristic of the illustrative examples are set forth in the appended claims. The illustrative examples, however, as well as a preferred mode of use, further objectives and descriptions thereof, will best be understood by reference to the following detailed description of an illustrative example of the present disclosure when read in conjunction with the accompanying drawings, wherein:

FIG. 1 depicts a simplified block diagram of a balloon dilation device, according to an example.

FIG. 2A depicts a perspective view of the balloon dilation device shown in FIG. 1, according to an example.

FIG. 2B depicts a perspective view of the balloon dilation device shown in FIG. 1, according to an example.

FIG. 3A depicts a side view of a shaft of a balloon dilation device, according to an example.

FIG. 3B depicts a cross-sectional view of a portion of the shaft shown in FIG. 3A, according to an example.

FIG. 4A depicts a side view of a shaft of a balloon dilation device, according to an example.

FIG. 4B depicts a cross-sectional view of a portion of the shaft shown in FIG. 4A, according to an example.

FIG. 5A depicts a portion of a shaft of a balloon dilation device, according to an example.

FIG. 5B depicts a cross-sectional view of the portion of the shaft shown in FIG. 5A, according to an example.

FIG. 5C depicts a partial assembly view of the shaft shown in FIG. 5A, according to an example.

FIG. 6A depicts a cross-sectional view of a portion of a shaft of a balloon dilation device, according to another example.

FIG. 6B depicts a subassembly of a portion of a position sensor of the shaft shown in FIG. 6A, according to an example.

FIG. 7 depicts a portion of a shaft and an inflatable balloon of a balloon dilation device, according to another example.

FIG. 8 depicts a cross-sectional view of a portion of the shaft of a balloon dilation device, according to another example.

FIG. 9 depicts a cross-sectional view of a portion of the shaft of a balloon dilation device, according to another example.

FIG. 10A depicts a side view of a balloon dilation device, according to another example.

FIG. 10B depicts a cross-sectional view of the balloon dilation device shown in FIG. 10A, according to an example.

FIG. 10C depicts an expanded view of a portion of the balloon dilation device shown in FIG. 10B, according to an example.

FIG. 10D depicts a side view of the balloon dilation device shown in FIG. 10A with a portion of a handle removed to show a plurality of internal components in an internal cavity of the handle, according to an example.

FIG. 10E depicts a distal portion of a shaft and a light fiber of the balloon dilation device shown in FIG. 10A, according to an example.

FIG. 10F depicts a cross-sectional view of the distal portion of the shaft shown in FIG. 10E.

FIG. 10G depicts an enlarged view of a portion of the cross-sectional view shown in FIG. 10F, according to the example.

FIG. 10H depicts a rear view of the dilation device shown in FIG. 10A, according to an example.

FIG. 10I depicts an assembly of a light source, a power source, and a light fiber for the balloon dilation device shown in FIG. 10A, according to an example.

FIG. 10J depicts an exploded view of the assembly of the light source, the power source, and the light fiber shown in FIG. 10I, according to an example.

FIG. 10K depicts a perspective view of a clip and a light fiber for the balloon dilation device shown in FIG. 10A, according to an example.

FIG. 10L depicts a cross-sectional view of the clip and the light fiber taken through an axis in FIG. 10K, according to an example.

FIG. 10M depicts a proximal portion of the light fiber shown in FIG. 10K, according to an example.

FIG. 10N depicts an arrangement for coupling one or more signal leads to a wire coil, according to an example.

FIG. 10O depicts an arrangement for coupling one or more signal leads to a wire coil, according to another example.

FIG. 11A depicts a perspective view of a balloon dilation device, according to another example.

FIG. 11B depicts a distal portion of a hypotube of the balloon dilation device shown in FIG. 11A, according to the example.

FIG. 11C depicts a cross-section view of the distal portion of the hypotube 116 shown in FIG. 11B, according to the example.

FIG. 12A depicts a side view of a shaft of a balloon dilation device, according to another example.

FIG. 12B depicts a cross-sectional view of a portion of the shaft shown in FIG. 12A, according to an example.

FIG. 13A depicts a bending tool, according to an example.

FIG. 13B depicts a hypotube in a bend channel of the bending tool shown in FIG. 13A, according to an example.

FIG. 13C depicts a first stage of a bending operation or a second stage of an unbending operation using the bending tool of FIG. 13A, according to an example.

FIG. 13D depicts a second stage of the bending operation or a first stage of an unbending operation shown in FIG. 13C, according to an example.

FIG. 13E depicts an unbending operation using a straightening channel of the bending tool shown in FIG. 13A, according to an example.

FIG. 14 depicts a flowchart for a process of forming a balloon dilation device is shown according to an example.

FIG. 15 depict additional aspects of the process shown in FIG. 14 according to another example.

FIG. 16 depicts a flowchart for a process of using a balloon dilation device is shown according to an example.

FIG. 17 depicts a flowchart for a process of forming a balloon dilation device is shown according to an example.

FIG. 18 depicts a flowchart for a process of using a balloon dilation device is shown according to an example.

### DETAILED DESCRIPTION

Disclosed examples will now be described more fully hereinafter with reference to the accompanying drawings, in which some, but not all of the disclosed examples are shown. Indeed, several different examples may be described and should not be construed as limited to the examples set forth herein. Rather, these examples are described so that this disclosure will be thorough and complete and will fully convey the scope of the disclosure to those skilled in the art.

By the term “approximately” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

As noted above, it may be beneficial to use image guided surgery while perform a procedure using a balloon dilation device to treat a sinus and/or a Eustachian tube. A conventional approach to image guided surgery in connection with a balloon dilation device is to insert a sensor wire equipped with a sensor into a working lumen of the balloon dilation device. The position sensor wire can include a plurality of coils that can sense an electromagnetic field and responsively provide a signal indicative of a position of the position sensor within the balloon dilation device.

One disadvantage to the conventional approach described above is that the position sensor wire fully occupies the working lumen of the balloon dilation device, which makes the working lumen unavailable for providing other functionalities through the working lumen. For example, when the position sensor wire is not positioned in the working lumen, the working lumen of the balloon dilation device may be used for aspiration and/or irrigation of fluids in a nasal cavity. As another example, when the position sensor wire is not position in the working lumen, a light fiber can be positioned in the working lumen to assist in providing a visual indication of the position of the balloon dilation device. For instance, the light fiber can emit light, which can be visually observed through transdermal illumination from an exterior of the nasal cavity and/or visually observed by an endoscope positioned in the nasal cavity. As yet another example, when the position sensor wire is not in the working lumen, the working lumen can be left unoccupied to provide for ventilation through the balloon dilation device, which may be beneficial when dilating a Eustachian tube.

The present disclosure can address at least some of the drawbacks of the above-described conventional approach to using a balloon dilation device with image guided surgery. Within examples, a balloon dilation device can include a position sensor that does not occupy a working lumen of the balloon dilation device. As a result, the balloon dilation device can (i) provide a sensor signal indicative of a position of the balloon dilation device for use with image guided surgery and (ii) perform at least one secondary function selected from a group consisting of: (a) aspirating a fluid through a working lumen of the balloon dilation device, (b) irrigating a body cavity with a fluid supplied through the working lumen of the balloon dilation device, (c) emitting light from a light fiber disposed in the working lumen of the balloon dilation device, and (d) ventilating a Eustachian tube through the working lumen of the balloon dilation device.

In some examples, the balloon dilation device can include a handle having a proximal handle end and a distal handle end, a shaft extending distally from the distal handle end, and an inflatable balloon disposed about at least a portion of a distal portion of the shaft. The shaft can include a hypotube having a proximal end, a distal end, and a body portion between the proximal end and the distal end of the hypotube. The hypotube can include an inner surface and an outer surface. The inner surface defines an inner diameter of the hypotube. The outer surface defines an outer diameter of the hypotube. The position sensor is coupled to a distal portion of the hypotube such that at least a portion of the position sensor is in a volume of space defined by (i) an inner radius that is equal to the inner diameter of the hypotube at the distal portion of the hypotube and (ii) an outer radius that is equal to the outer diameter of the hypotube at the distal portion of the hypotube.

In other examples, the balloon dilation device includes a handle having a proximal handle end and a distal handle end. The balloon dilation device also includes a shaft extending distally from the distal handle end, and an inflatable balloon disposed about at least a portion of a distal portion of the shaft. The shaft includes a hypotube having an inner surface and an outer surface. The inner surface defines a lumen of the hypotube. The shaft also includes a liner tube disposed within the lumen of the hypotube, and a position sensor coupled to a distal portion of the hypotube. The position sensor is between the inner surface of the hypotube and the liner tube.

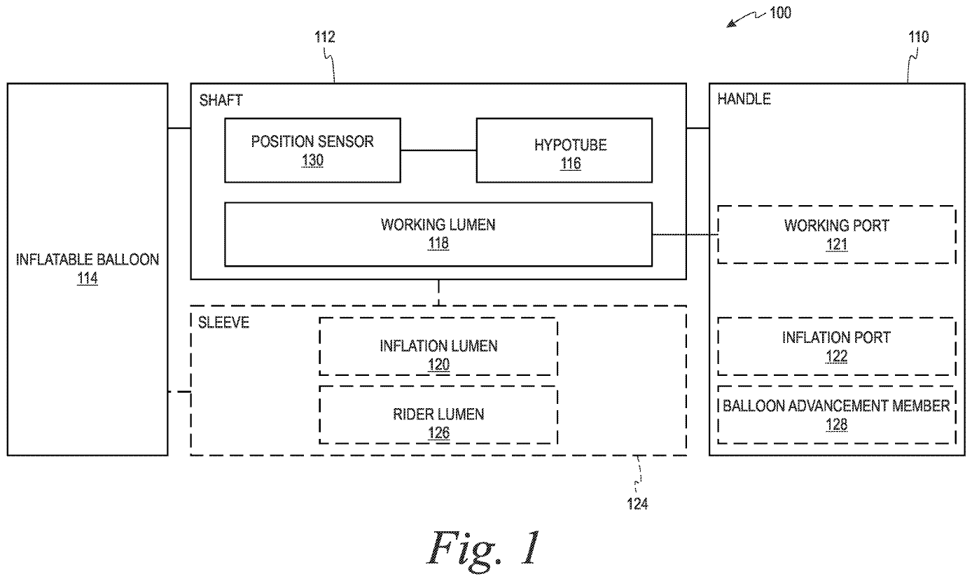

FIG. 1 shows a simplified block diagram of a balloon dilation device 100 according to an example. As shown in FIG. 1, the balloon dilation device 100 includes a handle 110, a shaft 112, and an inflatable balloon 114. The handle 110 has a proximal handle end and a distal handle end. In general, the handle 110 can be configured to facilitate a user gripping and manipulating the balloon dilation device 100 while performing a dilation procedure in a sinus ostium and/or a Eustachian tube. For example, the handle 110 can have a shape and/or a size that can facilitate a user performing the procedure by manipulating the balloon dilation device 100 using a single hand. In one implementation, the handle 110 can have a shape and/or a size that facilitates the user holding the balloon dilation device 100 using a writing utensil grip.

The handle 110 can define an internal cavity. As described in further detail below, the internal cavity can house one or more components of the balloon dilation device 100. In some implementations, the handle 110 is formed from a plurality of segments that are fixedly coupled together (e.g., by a welding coupling, an adhesive coupling, and/or a fastener coupling). This can help to assemble components within the internal cavity during a manufacturing process. However, in other implementations, the handle 110 can be formed as a single, monolithic structure that defines the internal cavity.

The shaft 112 extends distally from the distal handle end of the handle 110. For example, the shaft 112 can extend between a proximal end located in the internal cavity of the handle 110 and a distal end located external to the handle 110. The shaft 112 can be fixedly coupled to the handle 110 such that the shaft 112 and the handle 110 cannot move relative to each other.

The inflatable balloon 114 is disposed about at least a portion of a distal portion of the shaft 112. In this arrangement, the shaft 112 can help to provide underlying support for at least a portion of the inflatable balloon 114. For example, the shaft 112 can be formed from a substantially rigid material such as, for instance, a hypotube 116 (e.g., the hypotube 116 can be formed from stainless steel). Forming the shaft 112 from a substantially rigid material can additionally or alternatively assist in providing tactile feedback to a user holding the handle 110 while inserting the inflatable balloon 114 through a nasal cavity to a sinus ostium and/or the Eustachian tube.

In some examples, the shaft 112 can have a shape and/or a size that assists in navigating the distal end of the shaft 112 and the inflatable balloon 114 around anatomical structures of the patient while transnasally inserting the balloon dilation device 100 to the sinus ostium and/or the Eustachian tube. For instance, a distal portion of the shaft 112 can include a curved segment that can help to navigate the distal end of the shaft 112 and the inflatable balloon 114 through the nasal cavity to the sinus ostium and/or the Eustachian tube.

In some implementations, the shaft 112 can be non-malleable at the distal portion, including at the curved segment. In such implementations, the shape of the shaft 112 is fixed and cannot be changed without compromising a structural integrity of the shaft 112 (e.g., without forming a kink in the shaft 112). This can assist in inhibiting or preventing a user from altering the shaft 112 to have a shape that is not well suited for accessing an outflow tract of a particular sinus cavity (e.g., a frontal sinus cavity, a maxillary sinus cavity, or a sphenoid sinus cavity) and/or the Eustachian tube, and/or to inhibit a user from using the balloon dilation device 100 for treating an anatomical structure other than an intended sinus cavity and/or the Eustachian tube. Additionally, making the shaft 112 non-malleable at the distal portion can help to discourage an operator from attempting to reshape the distal portion in a manner that may compromise the integrity of the inflatable balloon 114.

In other implementations, the shaft 112 can be malleable at the distal portion. This may allow a user to modify the shape of the distal portion according to a specific anatomy of a particular patient and/or adapt the balloon dilation device 100 for treating one or more anatomical structures (e.g., one or more anatomical structures selected from a group consisting of an ostium of a frontal sinus cavity, an ostium of a maxillary sinus cavity, an ostium of a sphenoid sinus cavity, and an Eustachian tube). In an example of an implementation in which the shaft 112 is malleable at the distal portion, a manufacturing process can include a step of annealing the distal portion of the shaft 112 to impart malleability.

In some examples in which the distal portion of the shaft 112 is malleable or non-malleable, the curved segment can be formed during the manufacturing process and prior to a user preparing to use the balloon dilation device 100. In such examples, the balloon dilation device 100 can be preset to perform the dilation procedure on an anatomical structure without any modifications to the shaft 112 by the user. In other examples in which the distal portion of the shaft 112 is malleable, the shaft 112 can be manufactured without the curved segment, and the user can form the curved segment by bending the distal portion of the shaft 112 prior to inserting the balloon dilation device 100 into the nasal cavity of the patient.

The shaft 112 can define a working lumen 118 that can extend between a proximal end and a distal end of the shaft 112. For instance, the working lumen 118 can extend entirely through the shaft 112 between a proximal end and a distal end of the shaft 112. The working lumen 118 can be configured to provide for at least one secondary function selected from a group consisting of: (i) providing a light fiber through the balloon dilation device to indicate a position of the light fiber and/or the balloon dilation device 100 through transdermal illumination and/or endoscopic visualization, (ii) supplying a fluid to irrigate a sinus cavity and/or a Eustachian tube, (iii) aspirating a sinus cavity and/or a Eustachian tube, and (iv) ventilating a Eustachian tube.

In some examples, the proximal end of the shaft 112 can be coupled to a working port 121. The working port 121 can be at the proximal housing end of the handle 110, and can define an aperture providing access to the working lumen 118. In this arrangement, the working port 121 can be in communication with the working lumen 118 defined by the shaft 112. In some implementations, the working port 121 can include a fitting for coupling the working port 121 to a second device (e.g., a fluid source, a vacuum source, and/or a light source). As examples, the fitting can include a bayonet connection, a threaded connection, and/or a friction-fit connection.

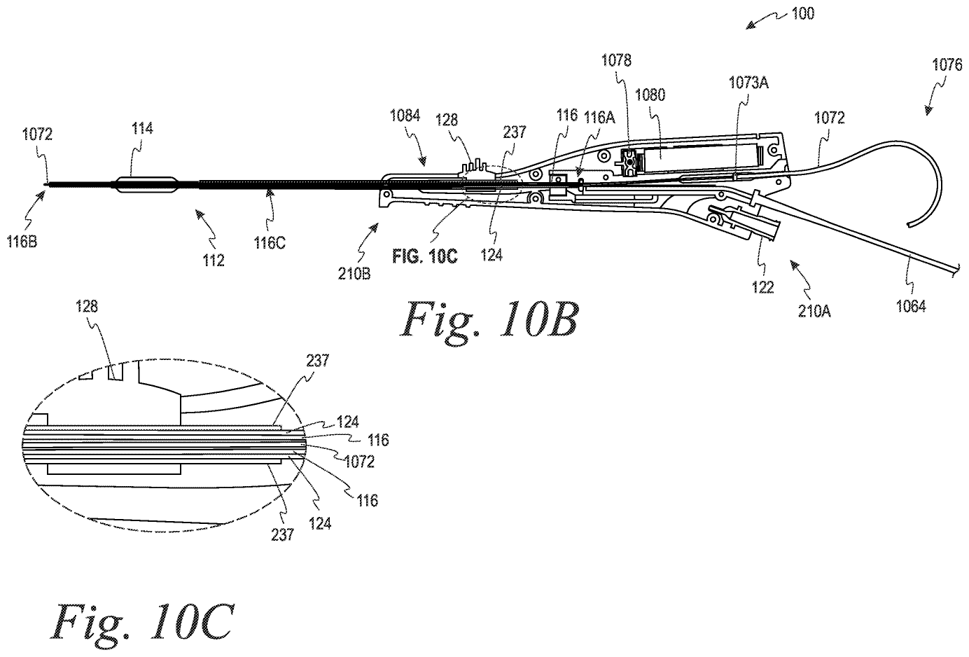

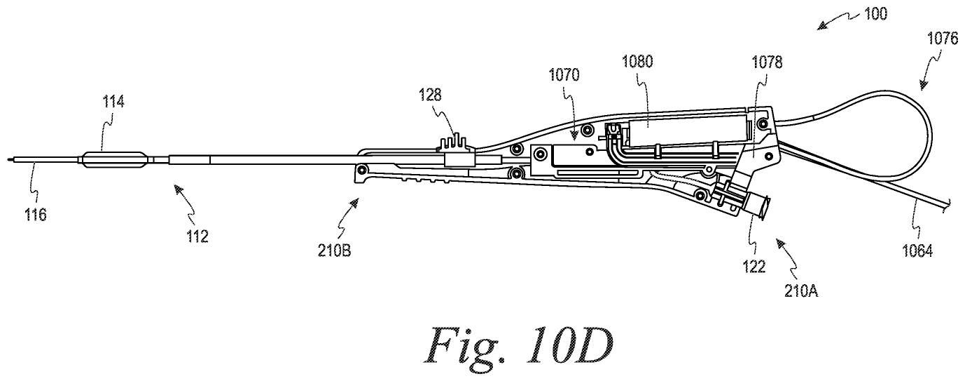

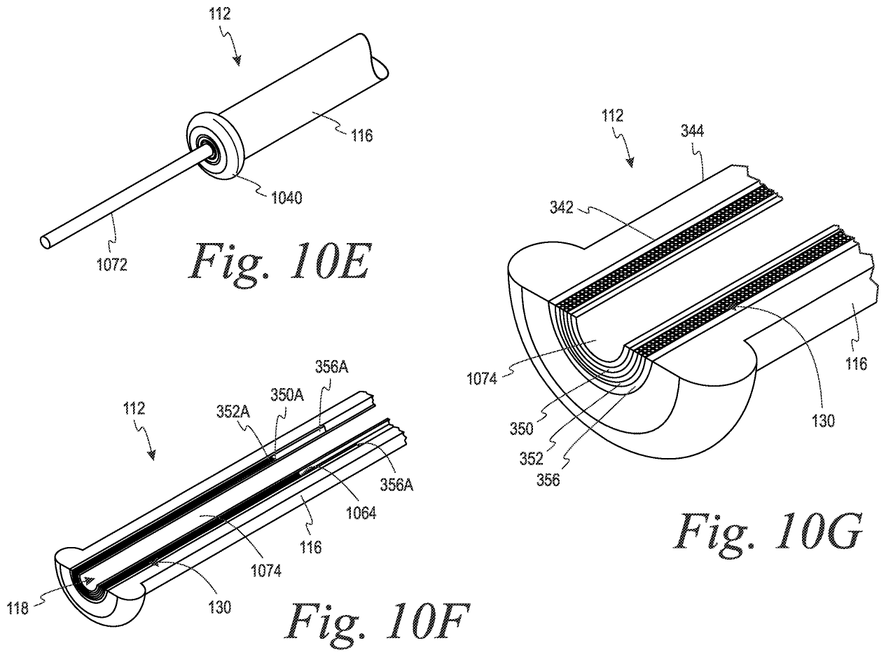

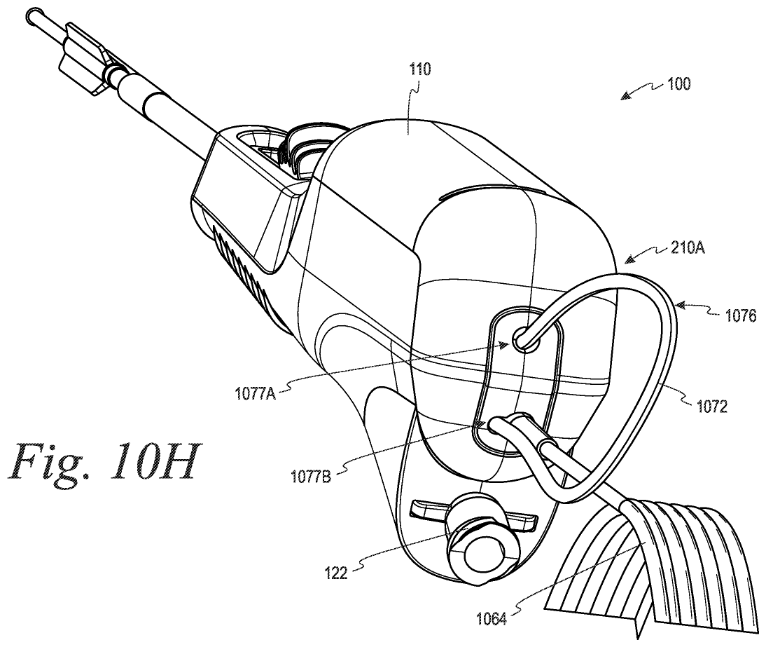

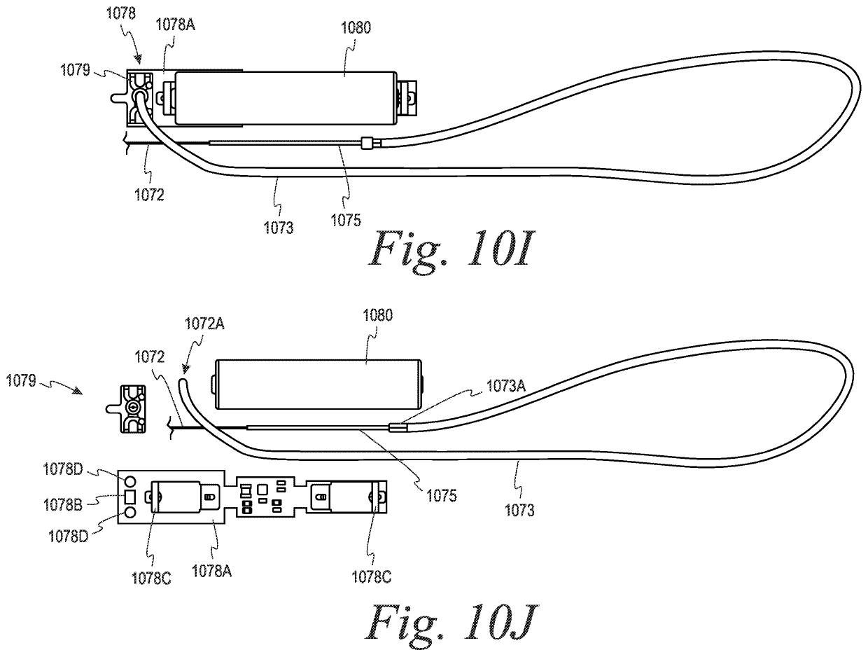

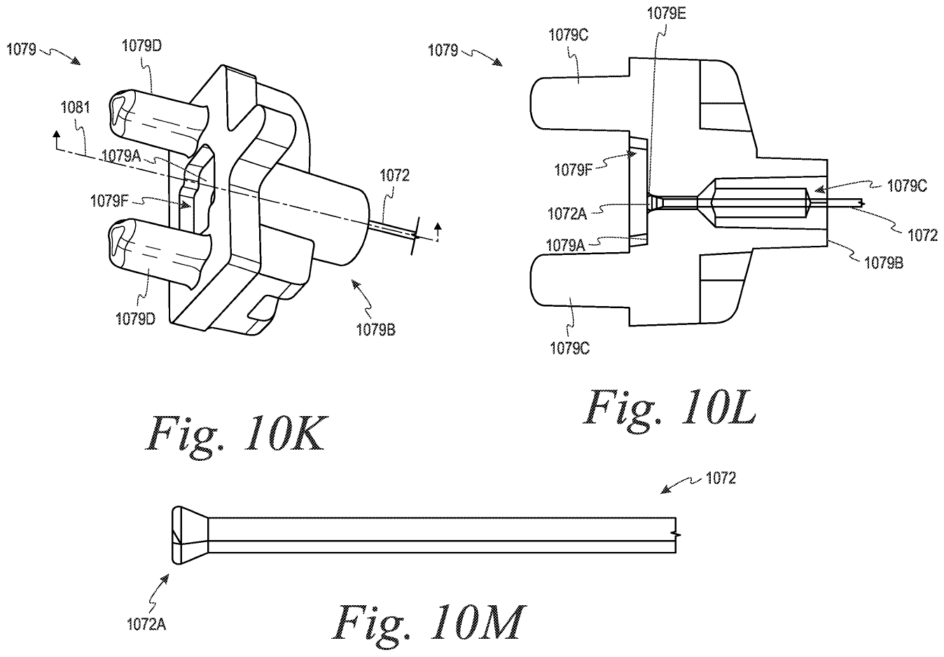

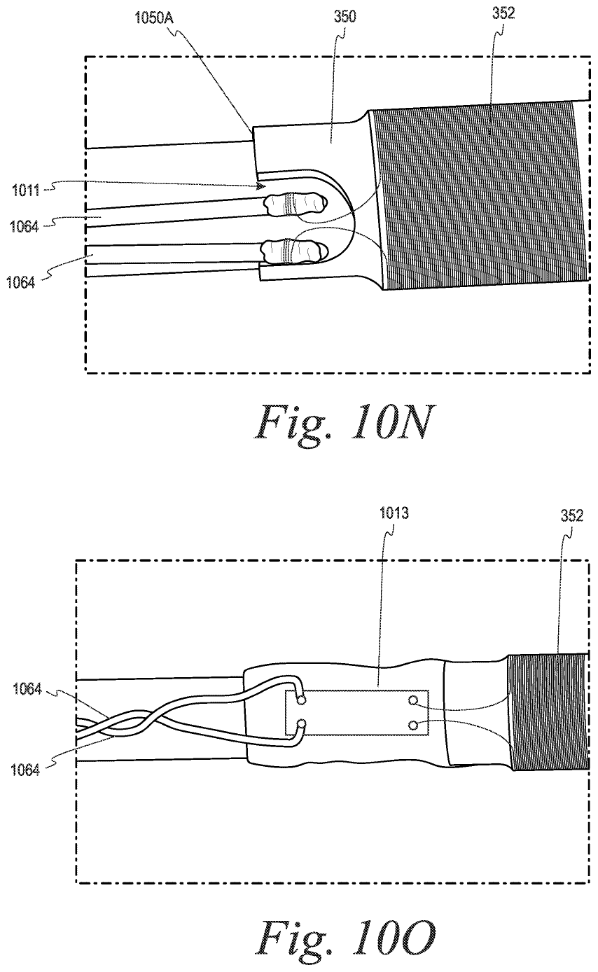

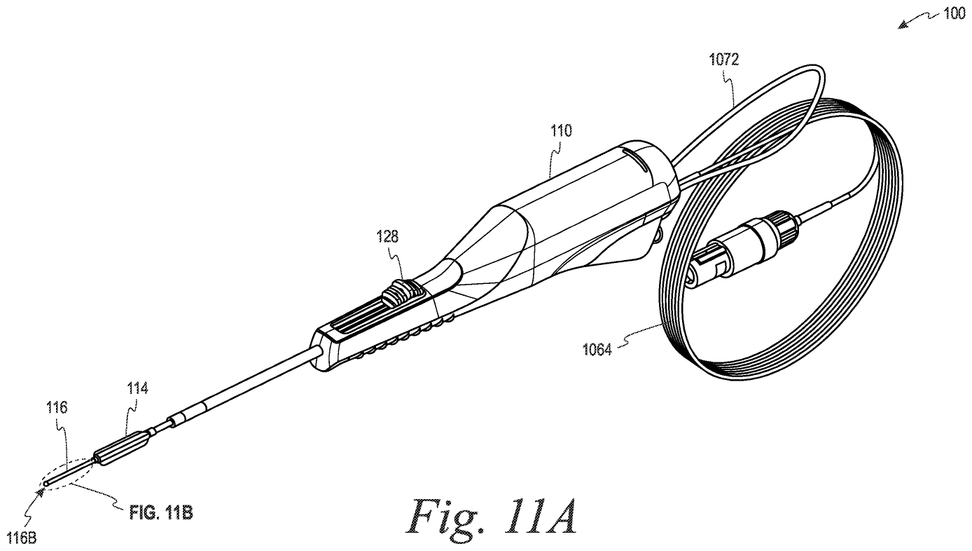

In other examples, the proximal end of the shaft 112 can be disposed in the internal cavity of the handle 110 such that the working lumen 118 cannot be accessed from an exterior of the handle 110. As described in further detail below with respect to FIGS. 10A-10G, this may be beneficial in an implementation in which a light fiber is non-removably disposed in the working lumen 118.

The distal end of the shaft 112 can define an aperture. This can help to provide for the at least one secondary function (e.g., providing a light fiber through the balloon dilation device to indicate a position of the light fiber and/or the balloon dilation device 100 through transdermal illumination and/or endoscopic visualization, supplying a fluid to irrigate a sinus cavity and/or a Eustachian tube, aspirating a sinus cavity and/or a Eustachian tube, and/or ventilating a Eustachian tube).

As described above, the inflatable balloon 114 is disposed about at least a portion of a distal portion of the shaft 112. The inflatable balloon 114 is configured to be actuated between a contracted state and an expanded state. In an example, the inflatable balloon 114 can have a cylindrical shape when the inflatable balloon 114 is inflated to the expanded shape. This shape of the inflatable balloon 114 can help to increase (or maximize) an amount of contact between the inflatable balloon 114 and the anatomy (e.g., the ostium of the sinus cavity and/or the Eustachian tube) when the inflatable balloon 114 is inflated to the expanded state. However, the inflatable balloon 114 can have a different shape in other examples.

In one example, the inflatable balloon 114 can have a cross-sectional diameter within the range of about 3 mm to about 9 mm when inflated to the expanded state. In another example, the inflatable balloon 114 can have a diameter within the range of about 5 to about 7 mm when inflated to the expanded state. In an example, the inflatable balloon 114 can have a length within the range of about 8 mm to 25 mm. These dimensions may be beneficial for treating the sinus cavities and/or Eustachian tube of most patients.

In some examples, the inflatable balloon 114 can be formed from a material having a flexibility that allows the inflatable balloon 114 to extend along and around the curved segment of the shaft 112. In some examples, the inflatable balloon 114 can be formed of one or more high strength and flexible polymeric materials such as, for instance, one or more polyamides (e.g., Nylon) and/or one or more elastomers (e.g., PEBAX). In one implementation of the manufacturing process, the inflatable balloon 114 can be “blow molded” to a relatively thin wall thickness, and capable of holding relatively high pressures from about 6 atmospheres to about 20 atmospheres of inflation pressure.

To actuate the inflatable balloon 114 between the contracted state and the expanded state, the balloon dilation device 100 can include an inflation lumen 120 extending from an inflation port 122 to the inflatable balloon 114. The inflation lumen 120 defines a fluid channel between the inflation port 122 and an interior of the inflatable balloon 114. The inflation lumen 120 is separate from the working lumen 118 of the shaft 112.

In some examples, the inflation port 122 can be coupled to the handle 110 as shown in FIG. 1. For instance, the inflation port 122 can be located at a proximal end of the handle 110. In other examples, the inflation port 122 can be separate from the handle 110. The inflation port 122 can include a connection interface for fluidically coupling with an inflation device such as, for instance, a syringe that can supply a fluid (e.g., saline or water) to the inflation port 122. As an example, the inflation port 122 can include a Luer connection interface for coupling the balloon dilation device 100 to the inflation device. In this arrangement, when the inflation device is coupled to the inflation port 122, the inflation device can be operated to (i) supply the fluid along the inflation lumen 120 to the inflatable balloon 114 and inflate the inflatable balloon 114 to the expanded state, and (ii) remove, along the inflation lumen 120, the fluid from the inflatable balloon 114 to deflate the inflatable balloon 114 to the contracted state.

In the example shown in FIG. 1, the inflation lumen 120 can be defined by a sleeve 124 having a first end that is fluidically coupled to the inflation port 122 and a second end that is fluidically coupled to the inflatable balloon 114. At least a distal section of the sleeve 124 can also include a rider lumen 126 that receives the shaft 112 such that the distal section of the sleeve 124 is disposed around the shaft 112. The inflation lumen 120 and the rider lumen 126 can be parallel to each other at the distal section of the sleeve 124.

In other examples, the inflation lumen 120 can be separate from the sleeve 124. For instance, the sleeve 124 can include only the rider lumen 126 and the sleeve 124 can extend around both the shaft 112 and a separate tube defining the inflation lumen 120. In this arrangement, the sleeve 124 can help to hold the inflation lumen 120 and the shaft 112 together.

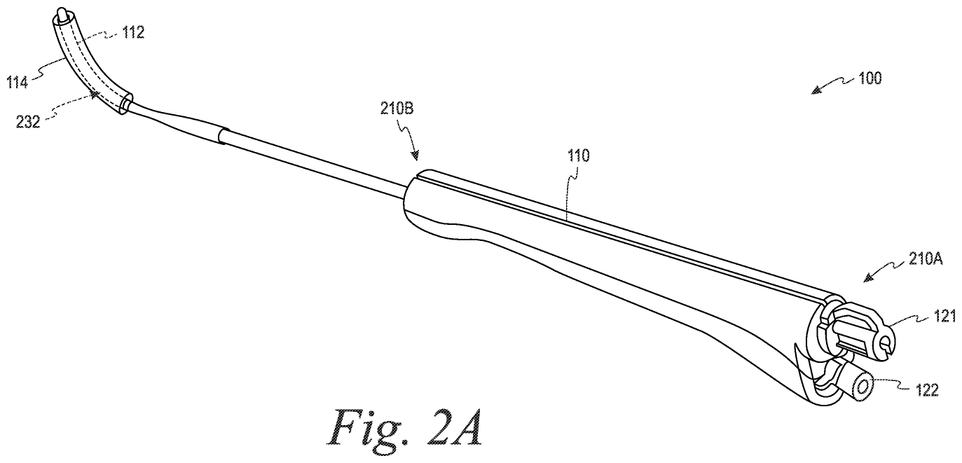

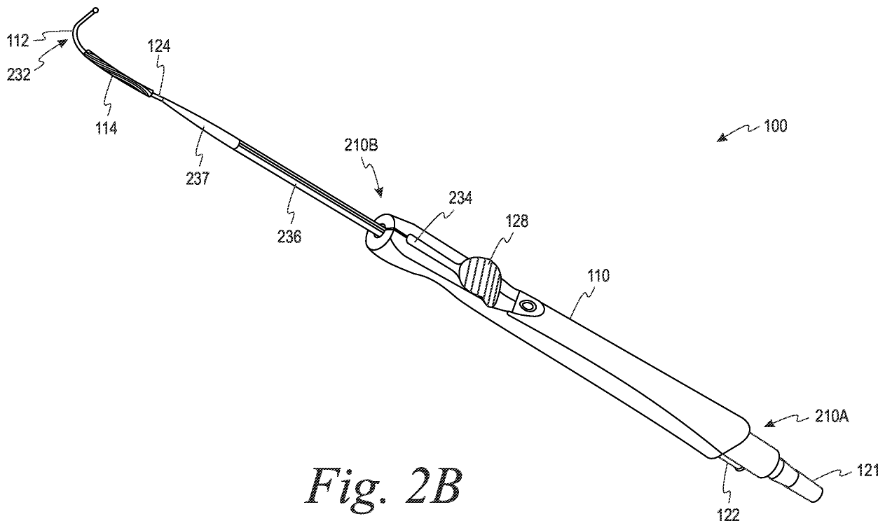

In some examples, the inflatable balloon 114 can be fixedly coupled to the shaft 112 such that the inflatable balloon 114 is immovable relative to the shaft 112. This may simplify a manufacturing process and reduce costs. However, fixedly coupling the inflatable balloon 114 to the shaft 112 can make navigating the inflatable balloon 114 into the target anatomy (e.g., an ostium of a sinus cavity and/or the Eustachian tube) more challenging. As described in further detail below, FIG. 2A depicts an example implementation of the balloon dilation device 100 in which the inflatable balloon 114 is fixedly coupled to the shaft 112.

In other examples, the inflatable balloon 114 can be movable relative to the shaft 112. This can help with inserting the inflatable balloon 114 into the target anatomy by allowing for (i) initially navigating the distal end of the shaft 112 and the inflatable balloon 114 to an entrance to a sinus ostium and/or the Eustachian tube opening in the nasopharynx using the handle 110 and (ii) then moving the inflatable balloon 114 relative to the shaft 112 to insert the inflatable balloon 114 through the sinus ostium and/or the Eustachian tube opening and to a position in the ostium of the sinus cavity and/or the Eustachian tube at which the inflatable balloon 114 can be actuated to the expanded state to dilate the ostium of the sinus cavity and/or the Eustachian tube. As described in further detail below, FIG. 2B depicts an example implementation of the balloon dilation device 100 in which the inflatable balloon 114 is movable relative to the shaft 112.

As shown in FIGS. 1 and 2B, the balloon dilation device 100 can include a balloon advancement member 128 that is configured to move the inflatable balloon 114 relative to the shaft 112. For example, the balloon advancement member 128 can be coupled to the sleeve 124, and the sleeve 124 can be coupled to the inflatable balloon 114. In this arrangement, moving the balloon advancement member 128 in a distal direction relative to the handle 110 moves the sleeve 124 and the inflatable balloon 114 distally relative to the shaft 112. Similarly, moving the balloon advancement member 128 in a proximal direction relative to the handle 110 moves the sleeve 124 and the inflatable balloon 114 proximally relative to the shaft 112. In this way, the inflatable balloon 114 is configured to move relative to the shaft 112 responsive to movement of the balloon advancement member 128 relative to the handle 110.

In some examples, the balloon advancement member 128 can be directly coupled to the sleeve 124. In other examples, the balloon advancement member 128 can be coupled to the sleeve 124 by a support tube such that the moving the balloon advancement member 128 moves the support tube, which moves the sleeve 124. The support tube and the sleeve 124 may move as a unit with movement of the balloon advancement member 128. The support tube can be located about the external periphery of a portion of the sleeve 124 to impart further stiffness to the balloon dilation device 100. For example, the support tube can be formed for a metallic material such as, for instance, a stainless steel hypotube.

In some implementations, the inflatable balloon 114 can be fixedly coupled to the shaft 112 at a position that is distal of a distal end of the shaft 112, or movable relative to the shaft 112 such that a distal end of the inflatable balloon 114 can be positioned distal of the distal end of the shaft 112. This may be beneficial in an implementation in which the balloon dilation device 100 is used to treat the Eustachian tube. For instance, this can facilitate inserting the inflatable balloon 114 into the Eustachian tube with a lesser portion (or no portion) of the shaft 112 extending into the Eustachian tube. Due to the relative flexibility and softness of the inflatable balloon 114 when in the contracted state as compared to the relatively rigid hardness of the shaft 112, this can help position the balloon dilation device 100 in the Eustachian tube in a more atraumatic manner than implementations in which at least a portion of the inflatable balloon 114 does not extend distally beyond the distal end of the shaft 112.

In other implementations, the inflatable balloon 114 can be fixedly coupled to the shaft 112 with the distal end of the inflatable balloon 114 positioned proximal of the distal endo the shaft 112, or the inflatable balloon 114 can be stopped from moving past the distal end of the shaft 112. This may help to enhance a tactile feedback provided by the balloon dilation device (e.g., in a manner similar to a seeker device) while navigating through a nasal cavity to an ostium of a sinus cavity and/or the Eustachian tube.

In some examples, the balloon dilation device 100 can additionally include an atraumatic tip at a distalmost end of the shaft 112 or the inflatable balloon 114. The atraumatic tip can help to provide for smooth movement of the balloon dilation device 100 through tight passages in the nasal cavity, and/or assist in providing tactile feedback to the user while navigating the inflatable balloon 114 to the ostium of the sinus cavity and/or the Eustachian tube. As examples, the atraumatic tip can have a bulbous shape and/or a bullet shape. In an example, a cross-section of the atraumatic tip can have an outer diameter in a range between about 1 mm and about 3 mm.

In some examples in which the atraumatic tip is at the distal end of the shaft 112, the atraumatic tip and the shaft 112 can be formed as a single, monolithic structure. In other examples in which the atraumatic tip is at the distalmost end of the inflatable balloon 114, the atraumatic tip and the inflatable balloon 114 are integrally formed as a single, monolithic structure. For instance, the atraumatic tip can be formed from excess material of the inflatable balloon 114 before, during, or after forming the inflatable balloon 114. Also, in examples in which the atraumatic tip is at the distal end of the inflatable balloon 114, the atraumatic tip can extend distally of the distal end of the shaft 112. In this arrangement, the atraumatic tip can provide a cushion between the distal end of the shaft 112, which can be made of a substantially rigid material, and the anatomy of the patient.

Although it can be beneficial to form the atraumatic tip and the inflatable balloon 114 as a single, monolithic structure, the atraumatic tip and the inflatable balloon 114 can be distinct structures that are coupled together in other examples. Similarly, in some examples in which the atraumatic tip is at the distalmost end of the shaft 112, the atraumatic tip and the shaft 112 can be distinct structures that are coupled together.

In some implementations in which the inflatable balloon 114 defines a distalmost end of the balloon dilation device 100, the inflatable balloon 114 can define a balloon lumen that can communicate with the working lumen 118. This can provide a continuous passageway from the distalmost end of the balloon dilation device 100 to the proximal end of the working lumen 118, which can help to provide the secondary function(s) using the working lumen 118 (e.g., providing a light fiber through the balloon dilation device to indicate a position of the light fiber and/or the balloon dilation device 100 through transdermal illumination and/or endoscopic visualization, supplying a fluid to irrigate a sinus cavity and/or a Eustachian tube, aspirating a sinus cavity and/or a Eustachian tube, and/or ventilating a Eustachian tube). Similarly, in some implementations in which the distal end of the shaft 112 defines the distalmost end of the balloon dilation device 100, the distal end of the shaft 112 can define an aperture such that the shaft 112 provides a continuous passageway from the distalmost end of the balloon dilation device 100 to the proximal end of the working lumen 118 to help provide the secondary function(s) described above.

As described above, the shaft 112 includes a position sensor 130 that is configured to sense an electromagnetic field and responsively generate, based on the electromagnetic field sensed by the position sensor 130, a signal that is indicative of a position of the position sensor 130. As shown in FIG. 1, the position sensor 130 is coupled to the hypotube 116. In particular, the position sensor 130 can be coupled to a distal portion of the hypotube 116 in a manner that does not occlude the working lumen 118 of the shaft 112. For instance, the position sensor 130 can include a through-bore, and the working lumen 118 can extend through the through-bore of the position sensor 130.

In some examples, at least a portion of the position sensor 130 can be in a volume of space defined by (i) an inner radius that is equal to the inner diameter of the hypotube 116 at the distal portion of the hypotube 116 and (ii) an outer radius that is equal to the outer diameter of the hypotube 116 at the distal portion of the hypotube 116. In other examples, the position sensor can be disposed between the hypotube 116 and an interior tube, and the interior tube can define the working lumen 118 of the shaft 112. Example arrangements of the hypotube 116, the position sensor 130, and the working lumen 118 are described in further detail below.

Referring now to FIG. 2A, a first implementation of the balloon dilation device 100 of FIG. 1 is shown according to one example. As shown in FIG. 2A, the balloon dilation device 100 includes the handle 110, the shaft 112, and the inflatable balloon 114. The handle 110 has a proximal handle end 210A and a distal handle end 210B, and the shaft 112 extends distally from the distal handle end 210B of the handle 110. The inflatable balloon 114 is disposed about at least a portion of a distal portion of the shaft 112. More particularly, in this example, the inflatable balloon 114 is fixedly coupled to the distal portion of the shaft 112 such that the inflatable balloon 114 is immovable relative to the shaft 112 and the handle 110.

In FIG. 2A, the distal portion of the shaft 112 includes a curved segment 232 that can help to navigate the distal end of the shaft 112 and the inflatable balloon 114 through the nasal cavity to the sinus ostium and/or the Eustachian tube. The distal portion of the shaft 112 can also include a straight segment that is distal of the curved segment 232. However, as described above, the distal portion of the shaft 112 can omit the curved segment 232 such that the shaft 112 is entirely straight and/or the distal portion of the shaft 112 can omit the straight segment distal of the curved segment 232.

As shown in FIG. 2A, the balloon dilation device 100 includes the working port 121 at the proximal handle end 210B, which provides access to the working lumen 118 (shown in FIG. 1) extending from the working port 121 and the distal end of the shaft 112. For example, the working port 121 can be coupled to the proximal end of the shaft 112 in the internal cavity of the handle 110. Additionally, as shown in FIG. 2A, the balloon dilation device includes the inflation port 122 at the proximal handle end 210A of the handle 110. As described above, the inflation port 122 is fluidly coupled to the inflation lumen 120 (shown in FIG. 1), which is fluidly coupled to the inflatable balloon 114.

Referring now to FIG. 2B, a second implementation of the balloon dilation device 100 of FIG. 1 is shown according to another example. As shown in FIG. 2A, the balloon dilation device 100 includes the handle 110, the shaft 112, and the inflatable balloon 114. The handle 110 has a proximal handle end 210A and a distal handle end 210B, and the shaft 112 extends distally from the distal handle end 210B of the handle 110. The inflatable balloon 114 is disposed about at least a portion of a distal portion of the shaft 112. More particularly, in this example, the inflatable balloon 114 is movable relative to the distal portion of the shaft 112.

As shown in FIG. 2B, the balloon dilation device 100 include the balloon advancement member 128 for moving the inflatable balloon 114 relative to the shaft 112 as described above. The balloon advancement member 128 can be configured enable a finger of the user (e.g., index finger or thumb) to easily move the balloon advancement member 128 relative to the handle 110. For example, as shown in FIG. 2B, the balloon advancement member 128 can include a plurality of protrusions that form a generally convex surface for engaging a finger of the user. The discontinuity between the protrusions can help to provide tactile feel and/or mitigate slippage of the finger on the balloon advancement member 128. However, the balloon advancement member 128 can have a different shape and/or a different size in other examples. Additionally, although the balloon advancement member 128 is moveable along a top surface of the handle 110 (e.g., a surface facing a direction in which the curved segment is bent) in the illustrated example, the balloon advancement member 128 can be disposed and moveable along a different surface of the handle 110 in other examples.

As shown in FIG. 2B, the balloon advancement member 128 can be configured to move within a slot 234 in the exterior surface of the handle 110. The slot 234 can extend in a direction that is parallel to a longitudinal axis of the shaft 112. In some examples, a length of the slot 234 can define a range of motion of the inflatable balloon 114 relative to the shaft 112. For instance, a distal end of the slot 234 can provide a distal stop to define a distalmost position of the inflatable balloon 114 relative to the shaft 112 and/or a proximal end of the slot 234 can define a proximal stop to define a proximal-most position of the inflatable balloon 114 relative to the shaft 112. In other examples, the slot 234 and the balloon advancement member 128 can be positioned in a recess of the handle 110, and the range of motion of the inflatable balloon 114 relative to the shaft 112 can be defined by a length of the recess and a length of the balloon advancement member 128. For instance, a distal edge of the balloon advancement member 128 can engage a distal edge of the recess when the inflatable balloon 114 is in the distalmost position, and a proximal edge of the balloon advancement member 128 can engage a proximal edge of the recess when the inflatable balloon 114 is in the proximal-most position. In still other examples, the range of motion of the inflatable balloon 114 can be defined by a combination of the length of the slot 234, the length of the recess, and/or the length of the balloon advancement member 128.

In FIG. 2B, the balloon advancement member 128 is coupled to the sleeve 124 by a support tube 236. The support tube 236 can extend along a proximal portion of the sleeve 124, but not a distal portion of the sleeve 124. This can help to achieve a desired flexibility of the distal portion of the shaft 112 and/or the distal portion of the sleeve 124, which may help to navigate the balloon dilation device 100 through the patient's anatomy during insertion. In other examples, the support tube 236 can extend over an entirety of the sleeve 124 between the handle 110 and the inflatable balloon 114.

As an example, as shown in FIG. 2B, the support tube 236 can be coupled to the sleeve 124 by a coupling 237. The coupling 237 can be a sheath having a proximal portion that extends around a distal portion of the support tube 236, and a distal portion that extends around the sleeve 124. In one implementation, the sheath can be melted or heat shrunk onto the support tube 236 and the sleeve 124. In the example shown in FIG. 2B, the balloon advancement member 128 is coupled to the support tube 236, the support tube 236 is coupled to the sleeve 124 by the coupling 237, and the sleeve 124 is coupled to the inflatable balloon 114. In other examples, the sleeve 124 can be directly coupled to the balloon advancement member 128 instead.

In FIG. 2B, the distal portion of the shaft 112 includes a curved segment 232 that can help to navigate the distal end of the shaft 112 and the inflatable balloon 114 through the nasal cavity to the sinus ostium and/or the Eustachian tube. However, as described above, the distal portion of the shaft 112 can omit the curved segment 232 such that the shaft 112 is entirely straight.

As shown in FIG. 2B, the balloon dilation device 100 includes the working port 121 at the proximal handle end 210B, which provides access to the working lumen 118 (shown in FIG. 1) extending from the working port 121 and the distal end of the shaft 112. For example, the working port 121 can be coupled to the proximal end of the shaft 112 in the internal cavity of the handle 110. Additionally, as shown in FIG. 2B, the balloon dilation device includes the inflation port 122 at the proximal handle end 210A of the handle 110. As described above, the inflation port 122 is fluidly coupled to the inflation lumen 120 (shown in FIG. 1), which is fluidly coupled to the inflatable balloon 114.

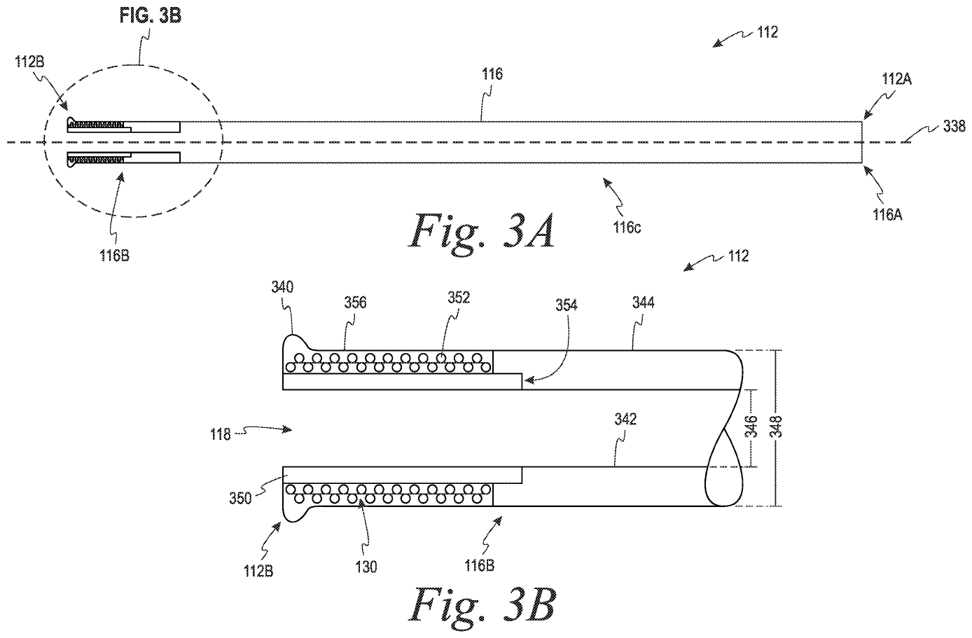

Referring now to FIGS. 3A-9, implementations of the shaft 112 are shown according to various examples. In the examples shown in FIGS. 3A-9, the shaft 112 includes the hypotube 116 and the position sensor 130 coupled to a distal portion of the hypotube 116. The hypotube 116 includes an inner surface defining an inner diameter of the hypotube 116, and an outer surface defining an outer diameter of the hypotube 116. At least a portion of the position sensor 130 is in a volume of space defined between (i) an inner radius that is equal to the inner diameter of the hypotube 116 at the distal portion of the hypotube 116 and (ii) an outer radius that is equal to the outer diameter of the hypotube 116 at the distal portion of the hypotube 116. For instance, the volume of space can be bounded by (i) the distal end of the hypotube 116, (ii) the distal end of the shaft 112, (iii) the outer radius equal to the outer diameter, and (iv) the inner radius equal to the inner diameter. In this arrangement, the position sensor 130 can be integrated into the shaft 112 without blocking the working lumen 118 of the shaft 112 so that working lumen 118 can be used for the secondary function(s) described above.

FIG. 3A depicts a side view of the shaft 112, and FIG. 3B depicts a cross-sectional view of a portion of the shaft 112 taken through a longitudinal axis 338 of the shaft 112 according to a first example. As shown in FIG. 3A, the shaft 112 extends between a proximal end 112A and a distal end 112B. In FIG. 3A, the shaft 112 includes an atraumatic tip 340 at the distal end 112B of the shaft 112. However, in other examples, the shaft 112 can omit the atraumatic tip 340.

As shown in FIGS. 3A-3B, the shaft 112 includes the hypotube 116 and the position sensor 130. The hypotube 116 has a proximal end 116A, a distal end 116B, and a body portion 116C between the proximal end 116A and the distal end 116B of the hypotube 116. In this example, the proximal end 116A of the hypotube 116 corresponds to the proximal end 112A of the shaft 112. As shown in FIG. 3B, the hypotube 116 also includes an inner surface 342 and an outer surface 344. The inner surface 342 defines an inner diameter 346 of the hypotube 116, and the outer surface 344 defines an outer diameter 348 of the hypotube 116.

Additionally, as shown in FIG. 3B, the position sensor 130 is coupled to a distal portion of the hypotube 116. At least a portion of the position sensor 130 is in a volume of space defined between (i) an inner radius that is equal to the inner diameter 346 of the hypotube 116 at the distal portion of the hypotube 116 and (ii) an outer radius that is equal to the outer diameter 348 of the hypotube 116 at the distal portion of the hypotube 116. For instance, the volume of space can be bounded by (i) the distal end 116B of the hypotube 116, (ii) the distal end 112B of the shaft 112, (iii) the outer radius equal to the outer diameter 348, and (iv) the inner radius equal to the inner diameter 346.

As shown in FIG. 3B, the position sensor 130 includes a ferromagnetic core 350 formed from a ferromagnetic alloy. In FIG. 3B, the ferromagnetic core 350 is in the form of a tube that extends from the distal end 116B of the hypotube 116. In this example, the position sensor 130 also includes a wire coil 352 wrapped around the ferromagnetic core 350. The position sensor 130 is configured such that an electromagnetic field supplied by an electromagnetic field generator at a surgical site induces a signal in the wire coil 352 when the position sensor 130 is positioned within the electromagnetic field. The strength and/or orientation of the signal is indicative of a position and an orientation of the position sensor 130. As such, the position sensor 130 can be configured to sense an electromagnetic field and, based on the electromagnetic field by the position sensor 130, generate a signal that is indicative of a six-dimensional position and orientation of the shaft 112 (e.g., a distal-most tip of the shaft 112).

Wrapping the wire coil 352 around the ferromagnetic core 350 can help to reduce a quantity of coils of the wire coil 352 and/or enhance a quality of the signal generated by the position sensor 130 responsive to the electromagnetic field at the surgical site. As an example, the ferromagnetic core 350 can be formed from mu metal. As another example, the ferromagnetic core 350 can be formed from metglas. In some examples, the ferromagnetic core 350 can include an electrically insulative coating on an exterior surface of the ferromagnetic core 350 that faces the wire coil 352 to help mitigate shorting between the ferromagnetic core 350 and the wire coil 352. As one example, the coating can include a parylene coating.

In this example, the ferromagnetic core 350 and the hypotube 116 define the working lumen 118 extending entirely through the shaft 112 between the proximal end 112A and the distal end 112B of the shaft 112. As shown in FIG. 3B, the ferromagnetic core 350 can have an inner diameter that is approximately equal to the inner diameter 346 of the hypotube 116. This can help to provide the working lumen 118 with a relatively constant diameter (e.g., equal to the inner diameter 346) over a length of the shaft 112, which can help to mitigate a resistance and/or damage to a light fiber extending and/or retracting through the working lumen 118.

In FIG. 3B, the ferromagnetic core 350 is coupled to the hypotube 116 at a recess 354 extending proximally from the distal end 116B of the hypotube 116. This can help to increase a surface area of contact between the ferromagnetic core 350 and the hypotube 116, which can help to strengthen a coupling between the ferromagnetic core 350 and the hypotube 116 (as compared to other examples in which the ferromagnetic core 350 is coupled to the distal end 116B of the hypotube 116). However, in other examples, the ferromagnetic core 350 can be coupled to the distal end 116B of the hypotube 116. This can simplify manufacture of the hypotube 116. As one example, the ferromagnetic core 350 can be coupled to the hypotube 116 by a weld at the interface between the ferromagnetic core 350 and the hypotube 116 (e.g., at the recess 354).

As shown in FIG. 3B, the wire coil 352 can be entirely between the inner surface 342 and the outer surface 344 of the hypotube 116 at the distal end 116B of the hypotube 116. This can help to reduce an overall size of the distal portion of the shaft 112, which may help improve a line of sight to the atraumatic tip 340 at the distalmost end of the shaft 112 (e.g., when using endoscopic visualization within a nasal cavity) and/or facilitate moving the inflatable balloon 114 over the distal portion of the shaft 112. However, in other examples, the wire coil 352 can extend radially outwardly of the outer surface 244 of the hypotube 116 (e.g., the wire coil 352 can have an outer diameter that is greater than the outer diameter 348 of the hypotube 116).

As shown in FIG. 3B, the shaft 112 can also include a protective cover 356 covering the wire coil 352. As an example, the protective cover 356 can include a heat shrink material such as, for example, a thermoplastic material (e.g., fluorinated ethylene propylene (FEP)). The protective cover 356 can help to retain and protect the wire coil 352.

In some examples, the protective cover 356 can extend from the distal end 116B of the hypotube 116 to the distal end 112B of the shaft 112. In some examples, the protective cover 356 can further extend proximally from the distal end 116B of the hypotube 116 toward the proximal end 116A of the hypotube 116. This can help to further enhance the protective cover 356 protecting and retaining the wire coil 352.

In some examples, the protective cover 356 has an outer diameter that is approximately equal to the outer diameter 348 of the hypotube 116 at the distal end 116B of the hypotube 116. This can help to reduce an overall size of the distal portion of the shaft 112, which may help improve a line of sight to the atraumatic tip 340 at the distalmost end of the shaft 112 (e.g., when using endoscopic visualization within a nasal cavity) and/or facilitate moving the inflatable balloon 114 over the distal portion of the shaft 112.

In FIGS. 3A-3B, the atraumatic tip 340 can be defined at least in part by the protective cover 356. For instance, a distal end of the protective cover 356 can have an outer diameter that is greater than an outer diameter of a proximal end of the protective cover such that the distal end of the protective cover 356 defines an atraumatic tip 340 of the shaft 112. However, in other examples, the outer diameter of the protective cover 356 can be approximately constant such that the shaft 112 omits the atraumatic tip 340 (e.g., the inflatable balloon 114 can include the atraumatic tip in other examples).

Although not shown in FIG. 3B, the position sensor 130 can further include one or more signal leads extending proximally from the wire coil 352 toward the handle 110. Within examples, the signal lead(s) can extend (i) through the working lumen 118, (ii) through the hypotube 116 (e.g., through a lumen extending parallel to the longitudinal axis 338 between the inner surface 342 and the outer surface 344), and/or (iii) outside of the outer surface 344 of the hypotube 116 (e.g., between the outer surface 344 and the sleeve 124). The signal lead(s) are configured to communicate the signal from the wire coil 352 to an image guided surgery device that can further process the signal and generate a display of the position and/or the orientation of the balloon dilation device relative to one or more images of anatomy.

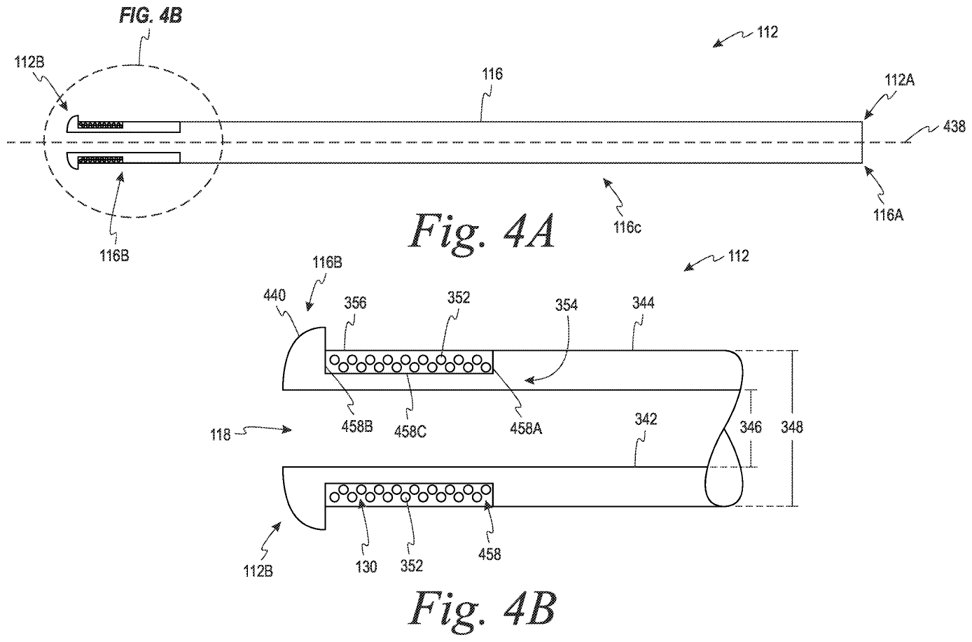

FIG. 4A depicts a side view of the shaft 112, and FIG. 4B depicts a cross-sectional view of a portion of the shaft 112 taken through a longitudinal axis 438 of the shaft 112 according to another example. As shown in FIG. 4A, the shaft 112 extends between the proximal end 112A and the distal end 112B. In FIG. 4A, the shaft 112 includes an atraumatic tip 440 at the distal end 112B of the shaft 112.

As shown in FIGS. 4A-4B, the shaft 112 includes the hypotube 116 and the position sensor 130. The hypotube 116 has the proximal end 116A, the distal end 116B, and the body portion 116C between the proximal end 116A and the distal end 116B of the hypotube 116. In this example, the proximal end 116A of the hypotube 116 corresponds to the proximal end 112A of the shaft 112. As shown in FIG. 4B, the hypotube 116 also includes the inner surface 342 and the outer surface 344. The inner surface 342 defines the inner diameter 346 of the hypotube 116, and the outer surface 344 defines the outer diameter 348 of the hypotube 116.

Additionally, as shown in FIG. 4B, the position sensor 130 is coupled to a distal portion of the hypotube 116. At least a portion of the position sensor 130 is in the volume of space defined between (i) an inner radius that is equal to the inner diameter 346 of the hypotube 116 at the distal portion of the hypotube 116 and (ii) an outer radius that is equal to the outer diameter 348 of the hypotube 116 at the distal portion of the hypotube 116.

As shown in FIG. 4B, the outer surface 344 of the hypotube 116 includes a recess 458 that extends radially towards the inner surface 342 of the hypotube 116. Additionally, as shown in FIG. 4B, the position sensor 130 can include a wire coil 352 wrapped around the hypotube 116 in the recess 458 of the hypotube 116. The recess 458 can include a proximal lateral wall 458A, a distal lateral wall 458B, and an axial wall 458C extending between the proximal lateral wall 458A and the distal lateral wall 458B. Along an axial dimension (e.g., the longitudinal axis 438), the wire coil 352 defines an outer diameter that is less than the outer diameter 348 of the hypotube 116 at the proximal lateral wall 458A. This can help to reduce an overall size of the distal portion of the shaft 112, which may help improve a line of sight to the atraumatic tip 440 at the distalmost end of the shaft 112 (e.g., when using endoscopic visualization within a nasal cavity) and/or facilitate moving the inflatable balloon 114 over the distal portion of the shaft 112 to the atraumatic tip 440.

As shown in FIG. 4B, the outer diameter 348 of the hypotube 116 is greater at the distal lateral wall 458B than the proximal lateral wall 458A. This can help to provide the atraumatic tip 440 at the distal end 116B of the hypotube 116. For instance, the outer diameter of a distal end 116B of the hypotube 116 can be greater than the outer diameter of a proximal portion of the hypotube 116 (e.g., a portion of the hypotube 116 that is proximal of the proximal lateral wall 458A) such that the distal end 116B of the hypotube 116 defines the atraumatic tip 440 of the shaft 112. However, in other examples, the outer diameter of a distal end 116B of the hypotube 116 can be approximately equal to the outer diameter of the proximal portion of the hypotube 116 such that the distal end 116B of the hypotube 116 such that the shaft 112 omits the atraumatic tip 340 (e.g., the inflatable balloon 114 can include the atraumatic tip in other examples).

In FIG. 4B, the shaft 112 also includes the protective cover 356 covering the wire coil 352 in the recess 458. The protective cover 356 can help to retain and protect the wire coil 352 in the recess 458. The protective cover 356 can extend over the recess 458 between the proximal lateral wall 458A and the distal lateral wall 458B. In some examples, the protective cover 356 can also extend proximally of the proximal lateral wall 458A and/or distally of the distal lateral wall 458B. This can help to further enhance the protective cover 356 protecting and retaining the wire coil 352.

In some examples, the protective cover 356 can have an outer diameter that is approximately equal to the outer diameter 348 of the hypotube 116 at a proximal lateral wall 458A of the recess 458. This can help to reduce an overall size of the distal portion of the shaft 112, which may help improve a line of sight to the atraumatic tip 440 at the distalmost end of the shaft 112 (e.g., when using endoscopic visualization within a nasal cavity) and/or facilitate moving the inflatable balloon 114 over the distal portion of the shaft 112.

In FIGS. 4A-4B, the distal end 116B of the hypotube 116 provides the atraumatic tip 440. However, in another example, the protective cover 356 can provide the atraumatic tip 440. For instance, the protective cover 356 can extend to the distal end 112B of the shaft 112, and a distal end of the protective cover 356 can have an outer diameter that is greater than an outer diameter of a proximal end of the protective cover 356 such that the distal end of the protective cover 356 defines an atraumatic tip of the shaft 112. In yet another example, the atraumatic tip 440 can be provided by both the distal end 116B of the hypotube 116 have an enlarged diameter and the protective cover 356 covering the distal end 116B of the hypotube 116.

As described above, the position sensor 130 is configured such that an electromagnetic field supplied by an electromagnetic field generator at a surgical site induces a signal in the wire coil 352 when the position sensor 130 is positioned within the electromagnetic field. The strength and/or orientation of the signal is indicative of a position and an orientation of the position sensor 130.

In FIG. 4B, the wire coil 352 is positioned on the hypotube 116 at the recess 358. However, in other examples, the position sensor 130 can also include a ferromagnetic core (e.g., the ferromagnetic core 350 shown and described below with respect to FIGS. 4A-4B) between the hypotube 116 and the wire coil 352. For instance, the wire coil 352 can be wrapped around the ferromagnetic core. The ferromagnetic core can be formed from a ferromagnetic material. For instance, the ferromagnetic core can be formed from at least one material selected from among a group consisting of: Mu metal and metglas. Additionally, as examples, the ferromagnetic core can have a form selected from a group consisting of: a foil, a tube, and a braid of wires. Disposing the wire coil 352 around the ferromagnetic core can help to reduce a quantity of coils of the wire coil 352 and/or enhance a quality of the signal generated by the position sensor 130 responsive to the electromagnetic field at the surgical site. As examples, the ferromagnetic core 350 can be formed from mu metal and/or metglas. Additionally, in an implementation in which the ferromagnetic core is formed from Mu metal, the Mu metal can have a form selected from a group consisting of: a foil formed from the Mu metal and a braid of wires formed from the Mu metal.

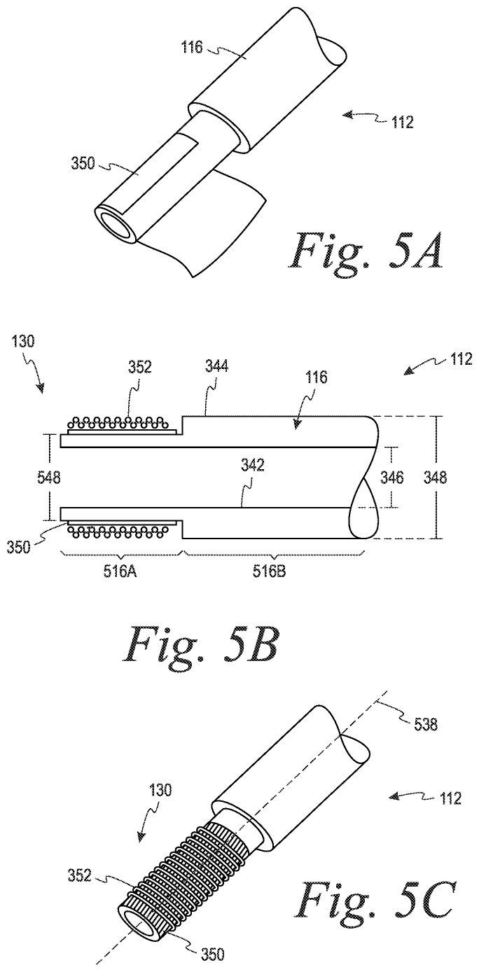

FIG. 5A depicts a portion of the shaft 112, FIG. 5B depicts a cross-sectional view of the portion of the shaft 112 taken through a longitudinal axis 538, and FIG. 5C depicts a partial assembly view of the shaft 112 according to another example.

As shown in FIGS. 5A-5C, the shaft 112 includes the hypotube 116 and the position sensor 130. As described above, the hypotube 116 has the proximal end 116A, the distal end 116B, and the body portion 116C between the proximal end 116A and the distal end 116B of the hypotube 116. As shown in FIG. 5B, the hypotube 116 also includes the inner surface 342 and the outer surface 344. The inner surface 342 defines the inner diameter 346 of the hypotube 116, and the outer surface 344 defines the outer diameter 348 of the hypotube 116.

Additionally, as shown in FIGS. 5A-5BB, the position sensor 130 is coupled to a distal portion of the hypotube 116. At least a portion of the position sensor 130 is in the volume of space defined between (i) an inner radius that is equal to the inner diameter 346 of the hypotube 116 at the distal portion of the hypotube 116 and (ii) an outer radius that is equal to the outer diameter 348 of the hypotube 116 at the distal portion of the hypotube 116.

In the implementation shown in FIGS. 5A-5C, a distal portion 516A of the hypotube 116 can include a reduced outer diameter relative to a proximal portion 516B of the hypotube 116. For instance, in FIGS. 5A-5C, the outer diameter 548 of the distal portion 516A of the hypotube 116 is less than the outer diameter 348 of the proximal portion 516B of the hypotube 116. In this example, the position sensor 130 includes the wire coil 352 on the outer surface 344 of the distal portion 516A of the hypotube 116. The reduced diameter of the distal portion 516A can thus provide the volume of space for positioning the position sensor 130, which may help improve a line of sight to the distalmost end of the shaft 112 (e.g., when using endoscopic visualization within a nasal cavity) and/or facilitate moving the inflatable balloon 114 over the distal portion of the shaft 112.

In FIG. 5B, the inner diameter 346 of the distal portion 516A is the same as the inner diameter 346 of the proximal portion 516B of the hypotube 116. This can help to provide a continuous surface within the working lumen 118, which can help to mitigate a light fiber snagging as the light fiber is inserted and/or retracted through the working lumen 118. In this arrangement, the distal portion 516A has a reduced thickness relative to the proximal portion 516B.

As shown in FIGS. 5A-5C, the position sensor 130 can also include a ferromagnetic core 350 between the hypotube 116 and the wire coil 352. As examples, the ferromagnetic core 350 can be formed from at least one material selected from among a group consisting of: Mu metal and metglas. Additionally, as examples, the ferromagnetic core 350 can have a form selected from a group consisting of: a foil, a tube, and a braid of wires. In FIGS. 5A-5C, the ferromagnetic core 350 is formed from the Mu metal, and the Mu metal has a form of a foil formed from the Mu metal. Disposing the wire coil 352 around the ferromagnetic core 350 can help to reduce a quantity of coils of the wire coil 352 and/or enhance a quality of the signal generated by the position sensor 130 responsive to the electromagnetic field at the surgical site.

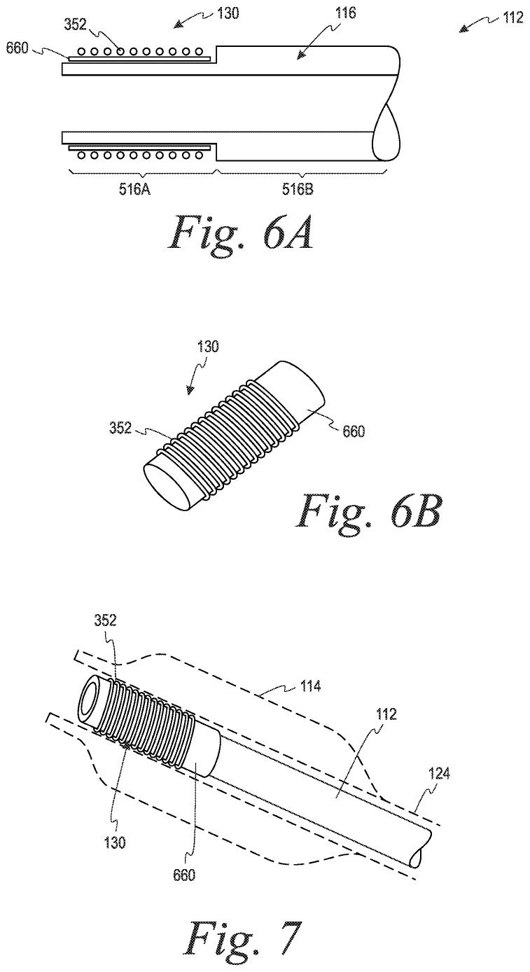

FIG. 6A depicts a cross-sectional view of a portion of the shaft 112 taken through a longitudinal axis (e.g., the longitudinal axis 538 in FIG. 5A), and FIG. 6B depicts a subassembly of a portion of the position sensor 130.

In FIGS. 6A-6B, the distal portion 516A of the hypotube 116 can include a reduced outer diameter relative to a proximal portion 516B of the hypotube 116 as described above with respect to FIGS. 5A-5B. Additionally, in FIGS. 6A-6B, the wire coil 352 is coupled to an exterior surface of a polymer tube 660, and the polymer tube 660 is configured to slide over the distal portion 516A of the hypotube 116 while the wire coil 352 is coupled to the exterior surface of the polymer tube 660. This can help to simplify a manufacture of the balloon dilation device 100. For instance, during the manufacturing process, the wire coil 352 can be coupled to the polymer tube 660 to form the subassembly shown in FIG. 6A, and then the subassembly can be slid over the distal portion 516A of the hypotube 116. Additionally, for instance, the polymer tube 660 can be configured to slide over the distal portion 516A of the hypotube 116 during an assembly process and can be fixedly coupled to the distal portion 516A of the hypotube 116. This can help to mitigate the position sensor 130 moving relative to the hypotube 116 in some implementations.

However, in other implementations, the polymer tube 660 and the position sensor 130 can be fixedly coupled to the inflatable balloon 114 instead of the hypotube 116. An example of one such implementation is shown in FIG. 7. As shown in FIG. 7, the polymer tube 660 is fixedly coupled to the inflatable balloon, the sleeve 124 is disposed around the shaft 112 and is configured to move the inflatable balloon 114 relative to the shaft 112, and the polymer tube 660 and the wire coil 352 are configured to move with the inflatable balloon 114 relative to the shaft 112.

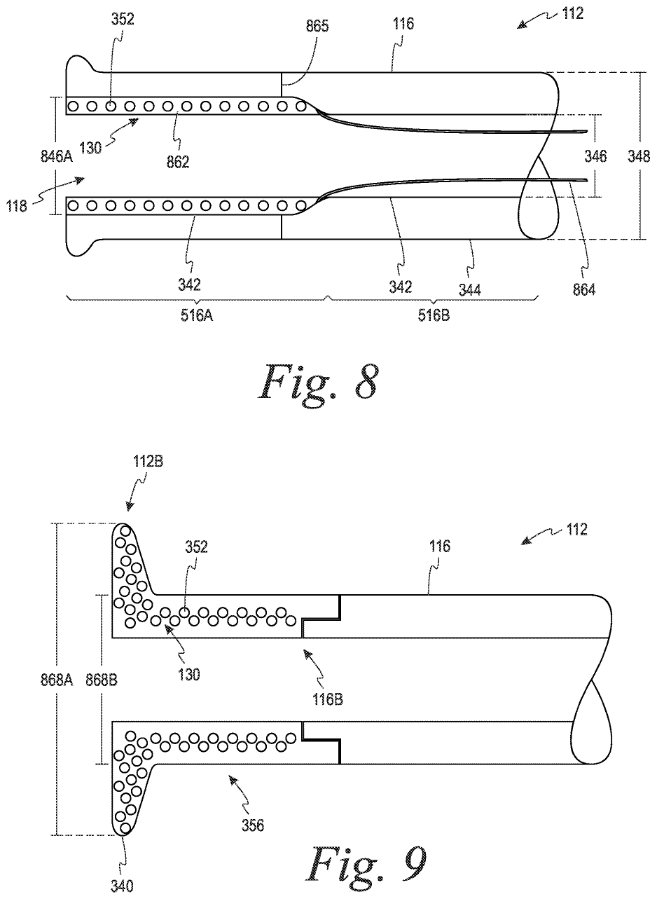

FIG. 8 depicts a cross-sectional view of a portion of the shaft 112 taken through a longitudinal axis of the shaft 112, according to another example. In the examples shown in FIG. 4A-7, the position sensor 130 is positioned radially outwardly of a portion of the hypotube 116. FIG. 8 shows an example implementation in which the position sensor 130 is positioned radially inwardly of a portion of the hypotube 116.

For example, in FIG. 8, an inner diameter 846A of the distal portion 516A of the hypotube 116 is greater than an inner diameter 846B of the proximal portion 516B of the hypotube 116. The position sensor 130 includes the wire coil 532 on the inner surface 342 of the distal portion 516A of the hypotube 116. In one example, the distal portion 516A of the hypotube 116 can include a counterbore formed in the distal end of the hypotube 116 to form the inner diameter 846A of the distal portion 516A and the inner diameter 846B of the proximal portion 516B of the hypotube 116. The distal portion 516A and the proximal portion 516B of the hypotube 116 can be formed as a single, integrated monolithic structure, or as separate components that are coupled to each other. In one example in which the distal portion 516A and the proximal portion 516B are separate components coupled to each other, the distal portion 516A of the hypotube 116 can be coupled to the proximal portion 516B of the hypotube 116 by a circumferential laser weld 865.

As shown in FIG. 8, the position sensor 130 can also include a support member 862 that can assist in coupling the wire coil 352 to the hypotube 116. In some examples, the support member 862 can be a polymer tube (e.g., the polymer tube 660 shown in FIGS. 6A-7. For instance, the wire coil 352 can be embedded in the polymer tube 660, and the polymer tube 660 can be coupled to the inner surface 342 of the distal portion 516A of the hypotube 116. In another example, the wire coil 352 can be disposed around the polymer tube 660 and the polymer tube 660 can be coupled to the inner surface 342 of the distal portion 516A of the hypotube 116 such that the wire coil 352 is disposed between the polymer tube 660 and the distal portion 516A of the hypotube 116.

In other examples, the support member 862 can be a ferromagnetic core (e.g., the ferromagnetic core 350 described above). For instance, the wire coil 352 can be between the ferromagnetic core 350 and the distal portion 516A of the hypotube 116. As described above, as examples, the ferromagnetic core can be formed from at least one material selected from among a group consisting of: Mu metal and metglas. Additionally, as examples, the ferromagnetic core can have a form selected from a group consisting of: a foil, a tube, and a braid of wires. Disposing the wire coil 352 around the ferromagnetic core can help to reduce a quantity of coils of the wire coil 352 and/or enhance a quality of the signal generated by the position sensor 130 responsive to the electromagnetic field at the surgical site. As examples, the ferromagnetic core 350 can be formed from mu metal and/or metglas.

As shown in FIG. 8, the position sensor 130 can further include one or more signal leads 864 extending proximally from the wire coil 352 toward the handle 110. In FIG. 8, the signal lead(s) 864 extend through the working lumen 118. However, in other examples, the signal lead(s) 864 can extend through the hypotube 116 (e.g., through a lumen extending parallel to the longitudinal axis between the inner surface 342 and the outer surface 344) and/or outside of the outer surface 344 of the hypotube 116 (e.g., between the outer surface 344 and the sleeve 124). As described above, the signal lead(s) 864 are configured to communicate the signal from the wire coil 352 to an image guided surgery device that can further process the signal and generate a display of the position and/or the orientation of the balloon dilation device 100 relative to one or more images of anatomy.

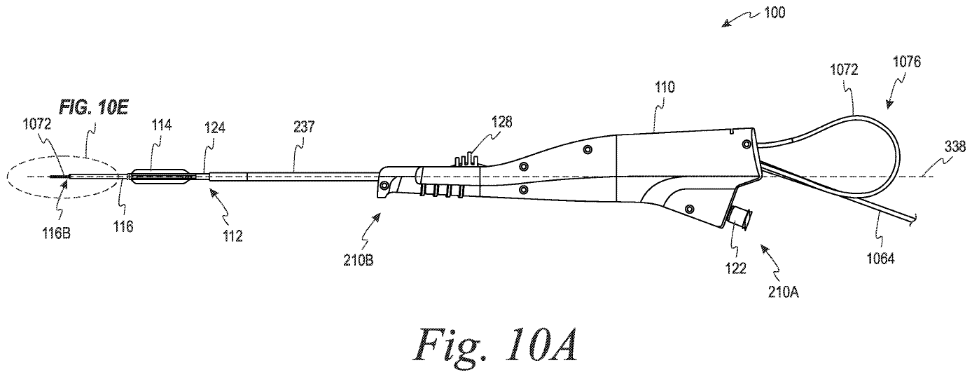

FIG. 9 depicts a cross-sectional view of a portion of the shaft 112 taken through a longitudinal axis of the shaft 112, according to another example. In FIG. 9, the shaft 112 includes a protective cover 356 that covers the wire coil 352 and is coupled to the distal end 116B of the hypotube 116. As such, the protective cover 356 extends to the distal end 112B of the shaft 112. In this example, a distal end of the protective cover 356 has an outer diameter 868A that is greater than an outer diameter 868B of a proximal end of the protective cover 356 such that the distal end of the protective cover 356 defines the atraumatic tip 340 of the shaft 112. As an example, the protective cover 356 can be formed from a polymer to provide a softer atraumatic tip 340 (e.g., as compared to implementations in which the atraumatic tip 340 is formed from a metal).

In FIG. 9, a portion of the wire coil 352 is disposed in the atraumatic tip 340 defined by the protective cover 356. This can help to increase a quantity and/or a density of coils of the wire coil 352 without increasing the portion of the shaft 112 that is proximal of the atraumatic tip 340. Additionally, as shown in FIG. 9, the portion of the wire coil 352 that is disposed in the atraumatic tip 340 can have an outer diameter that is greater than a portion of the wire coil 352 that is outside of the atraumatic tip 340. However, in other examples, the portion of the wire coil 352 that is disposed in the atraumatic tip 340 can have an outer diameter that is approximately equal to the portion of the wire coil 352 that is outside of the atraumatic tip 340. This can simplify a manufacturing process for forming the shaft 112.

Referring now to FIGS. 10A-10H, an implementation of the balloon dilation device 100 of FIG. 1 is shown according to another example. FIG. 10A depicts a side view of the balloon dilation device 100, FIG. 10B depicts a cross-sectional view of the balloon dilation device 100 taken through a longitudinal axis 338 of the shaft 112 (and with the inflation lumen 120 omitted for illustration purposes), FIG. 10C depicts an expanded view of a portion of the balloon dilation device 100 shown in FIG. 10B, FIG. 10D depicts a side view of the balloon dilation device 100 with a portion of the handle 110 removed to show a plurality of internal components in an internal cavity 1070 of the handle 110, FIG. 10E depicts a distal portion of the shaft 112 and a light fiber 1072, FIG. 10F depicts a cross-sectional view of the distal portion of the shaft 112 shown in FIG. 10E, FIG. 10G depicts an enlarged view of a portion of the cross-sectional view shown in FIG. 10F, and FIG. 10H depicts a rear view of the balloon dilation device 100, according to the example.

As shown in FIG. 10A, the balloon dilation device 100 includes the handle 110, the shaft 112, and the inflatable balloon 114. The handle 110 has a proximal handle end 210A and a distal handle end 210B, and the shaft 112 extends distally from the distal handle end 210B of the handle 110. The inflatable balloon 114 is disposed about at least a portion of a distal portion of the shaft 112.

The shaft 112 can include the hypotube 116 having the proximal end 116A, a distal end 116B, and the body portion 116C between the proximal end 116A and the distal end 116B of the hypotube 116. As shown in FIG. 10G, the hypotube 116 includes the inner surface 342 and the outer surface 344. The inner surface 342 defines a lumen of the hypotube 116. In some examples, the distal end 116B of the hypotube 116 can define an atraumatic tip 1040. However, in other examples, the shaft 112 can omit the atraumatic tip 1040.

Additionally, as shown in FIGS. 10F-10G, the shaft 112 can include a liner tube 1074 disposed within the lumen of the hypotube 116. For instance, the liner tube 1074 can be fixedly coupled to the hypotube 116. The shaft 112 includes the position sensor 130 coupled to a distal portion of the hypotube 116, and the position sensor 130 is between the inner surface 342 of the hypotube 116 and the liner tube 1074. For instance, as shown in FIGS. 10F-10G, the position sensor 130 can include the wire coil 352 disposed around the liner tube 1074 (and within the lumen of the hypotube 116). In this arrangement, the liner tube 1074 can define the working lumen 118 of the shaft 112 as shown in FIG. 10F.

In some examples, the liner tube 1074 can continuously extend along an entire length of the lumen of the hypotube 116 (e.g., the liner tube 1074 can be single, monolithic structure with a distal end of the liner tube 1074 at a distal end of the hypotube 116 and a proximal end of the liner tube 1074 at a proximal end of the hypotube 116). This can help to provide a continuous surface within the working lumen 118, which can help to mitigate a light fiber snagging as the light fiber 1072 is inserted and/or retracted through the working lumen 118. Although it can be beneficial to have liner tube 1074 continuously extend along the entire length of the lumen of the hypotube 116, the liner tube 1074 can have a different length than the hypotube 116 and/or the liner tube 1074 can be formed by a plurality of separate components coupled together and/or separated by gaps in other examples.

In addition to providing the working lumen 118, the liner tube 1074 can help to separate the wire coil 352 from the light fiber 1072 such that moving the light fiber 1072 in the working lumen 118 does not negatively affect the wire coil 352 (e.g., by mitigating forces that may impair the wire coil 352). The liner tube 1074 can additionally or alternatively assist in assembling the position sensor 130 in the lumen of the hypotube 116. In one example, the liner tube 1074 can be formed from a polymer material.

In some examples, the position sensor 130 can also include the ferromagnetic core 350 between the liner tube 1074 and the wire coil 352. As described above, this can help to reduce a quantity of coils of the wire coil 352 and/or enhance a quality of the signal generated by the position sensor 130 responsive to the electromagnetic field at the surgical site. However, in other examples, the position sensor 130 can omit the ferromagnetic core 350.

As shown in FIG. 10G, the shaft 112 can also include a protective cover 356 that covers the wire coil 352 between the wire coil 352 and the inner surface 342 of the hypotube 116. As an example, the protective cover 356 can include a heat shrink material such as, for example, a thermoplastic material (e.g., fluorinated ethylene propylene (FEP)). The protective cover 356 can help to retain and protect the wire coil 352 (e.g., help mitigate scratching the wire coil 352 and/or the hypotube 116). The protective cover 356 can additionally or alternatively help to electrically insulate the wire coil 352 from the hypotube 116.

As shown in FIG. 10F, a proximal end 356A of the protective cover 356 can be proximal of a proximal end 352A of the wire coil 352 and/or a proximal end of the ferromagnetic core 350A. This can help to further enhance the protective cover 356 retaining and protecting the wire coil 352.

Additionally, as shown in FIGS. 10A-10D and 10F, the balloon dilation device 100 can include one or more signal leads 1064 that extend from the wire coil 352 to a proximal end of the shaft 112. As described above, the signal lead(s) 1064 can communicate the signal from the wire coil 352 to an image guided surgery device that can further process the signal and generate a display of the position and/or the orientation of the balloon dilation device 100 relative to one or more images of anatomy. In FIG. 10F, the one or more signal leads 1064 are disposed between the liner tube 1074 and the inner surface 342 of the hypotube 116. As such, the liner tube 1074 can help to retain and protect the signal lead(s) 1064 outside of the working lumen 118. This can help to reduce snags or catch points between the light fiber 1072 and the signal lead(s) 1064. In other examples, the signal lead(s) can additionally or alternatively extend through the working lumen 118 (e.g., inside of the liner tube 1074) and/or the signal lead(s) 1064 can be embedded in the liner tube 1074.

As noted above, the liner tube 1074 can define the working lumen 118, and the working lumen 118 can extend from a distal end of the shaft 112 to a proximal end of the shaft 112. As shown in FIGS. 10A-10E, the light fiber 1072 is movable in the working lumen 118. This can allow a distalmost tip of the light fiber 1072 to extend and retract relative to the distal end of the shaft 112. In example shown in FIGS. 10A-10D, the light fiber 1072 can include a slack portion 1076 that extends out of the handle 110. The slack portion 1076 is configured to be inserted into the handle 110 and retracted out of the handle 110 to extend and retract, respectively, a distal portion of the light fiber 1072 from a distal end of the shaft 112.

As shown in FIG. 10H, the slack portion 1076 of the light fiber 1072 can extend through a first aperture 1077A and a second aperture 1077B in the proximal handle end 210A of the handle 110 such that the slack portion 1076 forms a loop. This can help to mitigate tangling of the slack portion 1076 with other cables and/or tubes (e.g., a cable including the signal lead(s) 1064 and/or an inflation device coupled to the inflation port 122).

Although the slack portion 1076 is external to the handle 110 in FIGS. 10A-10B and 10D, the slack portion 1076 can be disposed in the internal cavity 1070 of the handle 110 in other examples. For instance, the handle 110 can include a light fiber advancer member that can move relative to the handle 110 to responsively extend and retract the light fiber 1072 relative to the handle 110. As examples, the light fiber advancer member can include one or more of a group consisting of: a slider that is slidable along a slot extending parallel to the longitudinal axis 338 of the shaft 112, a rotatable wheel, and a rotatable dial.

As shown in FIG. 10B, the balloon dilation device 100 can also include a light source 1078 and/or a power source 1080 in an internal cavity 1070 defined by the handle 110. As examples, the light source 1078 can include one or more light emitting diodes (LEDs), organic light emitting diodes (OLEDs), optical fibers, non-fiber optic waveguides, and/or lenses. Additionally, as examples, the power source 1080 can include one or more batteries. The power source 1080 is electrically coupled to the light source 1078, and the light source 1078 is optically coupled to the light fiber 1072. In this arrangement, the power source 1080 can supply electrical power to the light source 1078, the light source 1078 can use the electrical power to emit light, the light fiber 1072 can transmit the light from the light source 1078 to the distal end of the light fiber 1072, and the light fiber 1072 can emit the light at the distal end of the light fiber 1072. The light source 1078 and the light fiber 1072 can be configured to emit the light with a power and/or an intensity such that the light emitted from the light fiber 1072 be visually observed through transdermal illumination from an exterior of the nasal cavity and/or visually observed by an endoscope positioned in the nasal cavity. In this way, the light fiber 1072 can help to confirm a position of the distal end of the light fiber 1072 (and/or the shaft 112) in the nasal cavity.

FIG. 10I depicts an assembly of the light source 1078, the power source 1080, and the light fiber 1072, and FIG. 10J depicts an exploded view of the assembly of the light source 1078, the power source 1080, and the light fiber 1072 shown in FIG. 10I according to an example. As shown in FIGS. 10I-10J, the balloon dilation device 100 can also include a fiber sheath 1073 that can cover a proximal portion of the light fiber 1072. The fiber sheath 1073 can extend along at least the slack portion 1076 of the light fiber 1072 shown in FIG. 10B and FIG. 10H. In such examples, the fiber sheath 1073 can improve handling and robustness of the light fiber 1072 at the slack portion 1076, which is external to the handle 110 as described above. As examples, the fiber sheath 1073 can be formed from at least one material selected from a group of materials consisting of: a fluoropolymer, silica, and low refractive index plastics (e.g., acrylate, propionate, acetate, and/or siloxane).

Additionally, as shown in FIGS. 10I-10J, the balloon dilation device 100 can include a support sleeve 1075 that extends along at least a portion of the light fiber 1072. For instance, in FIGS. 10B and 101-10J, the support sleeve 1075 can extend along at least an intermediate portion of the light fiber 1072 that extends from a distal end 1073A of the fiber sheath 1073 to the proximal end 116B of the hypotube 116. The support sleeve 1075 can provide structural support to the light fiber 1072 over a space in the internal cavity 1070 between the distal end 1073A of the fiber sheath 1073 and the proximal end 116B of the hypotube 116. As examples, the support sleeve 1075 can be formed from at least one material selected from a group of materials consisting of: polymide, polyetheretherketone (PEEK), acrylonitrile butadiene styrene (ABS), polycarbonate, polyethylene terephthalate (PET), fluorinated ethylene propylene (FEP, and nylon.

As also shown in FIGS. 10I-10J, the light source 1078 can include a light source circuit board 1078A and a light emitter 1078B. The light source circuit board 1078A includes a plurality of electrical contacts 1078C for electrically coupling the light emitter 1078B to the power source 1080. As described above, the light emitter 1078B can include, for example, one or more light emitting diodes (LEDs), and/or one or more organic light emitting diodes (OLEDs).

The light fiber 1072 is optically coupled to the light emitter 1078B of the light source 1078. For example, in FIGS. 10I-10J, a proximal end 1072A of the light fiber 1072 can be butt-coupled to the light emitter 1078B. To position the proximal end 1072A of the light fiber 1072 at the light emitter 1078B, the balloon dilation device 100 can include a clip 1079.

FIG. 10K depicts a perspective view of the clip 1079 and the light fiber 1072, FIG. 10L depicts a cross-sectional view of the clip 1079 and the light fiber 1072 taken through an axis 1081 in FIG. 10K, and FIG. 10M depicts a proximal portion of the light fiber 1072, according to an example. As shown in FIGS. 10K-10L, the clip 1079 can include a first end 1079A that is configured to face the light source 1078, and a second end 1079B that is opposite the first end 1079A. The clip 1079 can also include one or more clip couplers 1079D (shown in FIGS. 10K-10L) that are configured to couple to one or more light source couplers 1078D of the light source 1078 (shown in FIG. 10J). In the example shown in FIGS. 10J-10L, the one or more clip couplers 1079D include a plurality of protrusions, the one or more light source couplers 1078D include a plurality of apertures, and the protrusions are configured to be friction-fit coupled to the apertures. However, in other examples, the one or more clip couplers 1079D can include the apertures and the one or more clip couplers 1079D can include the protrusions. In still other examples, the clip 1079 and the light source 1078 can include be configured to couple to each other by at least one coupling selected from a group consisting of: a friction-fit coupling, an adhesive coupling, a threaded coupling, a snap-fit coupling, a heat stake coupling, and a welded coupling.

As shown in FIGS. 10K-10L, the clip 1079 can include a through-bore 1079C that extends between the first end 1079A and the second end 1079B of the clip 1079. The light fiber 1072 extends through the through-bore 1079C of the clip 1079. The proximal end 1072A of the light fiber 1072 can have a size that is greater than a diameter of the through-bore 1079C such that the light fiber 1072 is axially retained in the clip 1079. As shown in FIG. 10L, the proximal end 1072A of the light fiber 1072 can be substantially flush with the first end 1079A of the clip 1079. This can help to improve the butt-coupling between the proximal end 1072A of the light fiber 1072 and the light emitter 1078B.

As shown in FIGS. 10K-10M, the light fiber 1072 can include a flared shape at the proximal end 1072A of the light fiber 1072, which can help to position the proximal end 1072A of the light fiber 1072 at the first end 1079A of the clip 1079. For instance, a diameter of the light fiber 1072 can taper outwardly from the axis 1081 along a proximal direction towards the proximal end 1072A. The flared shape at the proximal end 1072A of the light fiber 1072 can also provide a fiber optic taper that can help to improve optical coupling efficiency and/or reduce losses due to scattering, reflection, and/or misalignment between the light source 1078 and the light fiber 1072. In some examples in which the light fiber 1072 includes the fiber sheath 1073, the fiber sheath 1073 can extend over the flared shape at the proximal end 1072A of the light fiber 1072.

In some examples, the clip 1079 can include a recess 1079E having a shape that corresponds to the flared shape at the proximal end 1072A of the light fiber 1072. For instance, the recess 1079E of the clip 1079 can have a taper (e.g., a conical shape) that matches the taper of the proximal end 1072A of the light fiber 1072. This can help to enhance alignment of the proximal end 1072A of the light fiber 1072 relative to the clip 1079 and the light source 1078. For instance, an engagement between the proximal end 1072A of the light fiber 1072 and the recess 1079E of the clip 1079 can help to self-center the proximal end 1072A and/or control a depth of the proximal end 1072A of the light fiber 1072 relative to the first end 1079A of the clip 1079.

As shown in FIGS. 10K-10L, the clip 1079 can also include a cavity 1079F that is configured to receive the light emitter 1078D. The cavity 1079F is adjacent to the first end 1079A. The cavity 1079F and the light emitter 1078D can have respective shapes and sizes such that the light emitter 1078D can extend into the cavity 1079F and abut against the first end 1079A of the clip 1079. This can help to butt-couple the proximal end 1072A of the light fiber 1072 with the light emitter 1078D.

As described above, the balloon dilation device 100 can be used to dilate a narrow or obstructed sinus passage or a Eustachian tube. In implementations in which the balloon dilation device 100 is used to treat the Eustachian tube, the inflatable balloon 114 can be inflated to dilate a Eustachian tube. When the inflatable balloon 114 is inflated, the inflatable balloon 114 may contact the Eustachian tube around a substantial portion or an entirety of a circumference of the inflatable balloon 114. In these instances, there may little or no space between an exterior of the inflatable balloon 114 and the Eustachian tube for air to flow through the Eustachian tube. If airflow was blocked while the inflatable balloon 114 is inflated in the Eustachian tube, pressure may build up at the middle ear and this may present a risk of potential damage to the tympanic membrane in the middle ear.

To mitigate this risk, the balloon dilation device 100 is configured to provide an airflow pathway through the balloon dilation device 100 while the inflatable balloon 114 is inflated and in contact with the Eustachian tube. In FIGS. 10A-10G, the balloon dilation device 100 includes the light fiber 1072 in the working lumen 118. However, the light fiber 1072 and the working lumen 118 can have respective diameters such that an air gap 1082 is formed between the light fiber 1072 and the working lumen 118. The air gap 1082 can define an airflow pathway between the distal end 112B of the shaft 112 and a vent opening 1084 in the handle 110. In this arrangement, the balloon dilation device 100 can provide the airflow pathway from a location in the Eustachian tube that is distal of the inflatable balloon 114 to a location of the handle 110 that is external to the patient's anatomy (e.g., the Eustachian tube and a nasal cavity).

As described above, the balloon dilation device 100 can also include the vent opening 1084 in an exterior surface of the handle 110 to further define the airflow pathway. As shown in FIG. 10B, the vent opening 1084 can be disposed between the proximal handle end 210A of the handle 110 and the distal handle end 210B of the handle 110. Additionally or alternatively, the vent opening 1084 can be nearer to the distal handle end 210B of the handle 110 than the proximal handle end 210A of the handle 110.

For instance, in FIG. 10B, the vent opening 1084 is defined by a slot in the handle 110 along which the balloon advancement member 128 is movable. This can reduce (or minimize) the number of openings in the exterior surface of the handle 110 as compared to other examples in which the vent opening 1084 includes one or more other openings providing only the venting functionality of the vent opening 1084. However, in other examples, the vent opening 1084 can include one or more other openings in the exterior surface of the handle 110 (e.g., in an implementation in which the balloon dilation device 100 omits the balloon advancement member 128 and/or the slot).

In the example shown in FIGS. 10A-10C, the airflow pathway can be defined by the air gap 1082 extending from the distal end of the shaft 112 (e.g., at the distal end 116B of the hypotube 116) to the proximal end 116A of the hypotube 116, the internal cavity of the handle 110, and the vent opening 1084. In this arrangement, while the inflatable balloon 114 is in the expanded state, air can flow through the distal end of the shaft 112 into the air gap 1082. The air can then flow proximally along the air gap 1082 between the light fiber 1072 and the liner tube 1074 and out the proximal end 116A of the hypotube 116 into the internal cavity of the handle 110. The air can then flow from the internal cavity out of the handle 110 through the vent opening 1084 to ventilate the Eustachian tube.

In FIGS. 10E-10G, a distal end of the liner tube 1074 is at a distal end of the hypotube 116. However, in other examples, the distal end of the liner tube 1074 can extend distally of the distal end of the hypotube 116. For instance, in some examples, the distal end of the liner tube 1074 can extend distally of the distal end of the hypotube 116 and define the atraumatic tip 1040 of the shaft 112.

As described above, the balloon dilation device 100 can include the one or more signal leads 1064 that extend from the wire coil 352 to a proximal end of the shaft 112, and the signal lead(s) 1064 can communicate the signal from the wire coil 352 to an image guided surgery device that can further process the signal and generate a display of the position and/or the orientation of the balloon dilation device 100 relative to one or more images of anatomy.

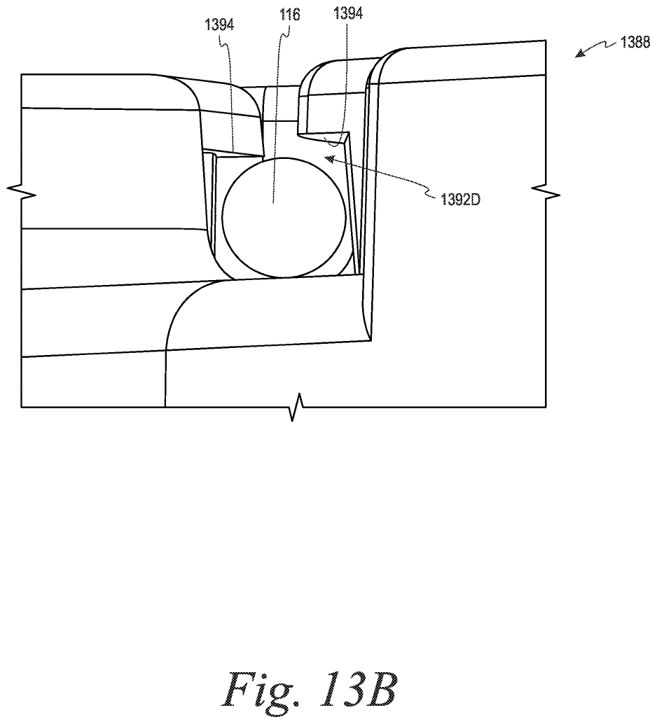

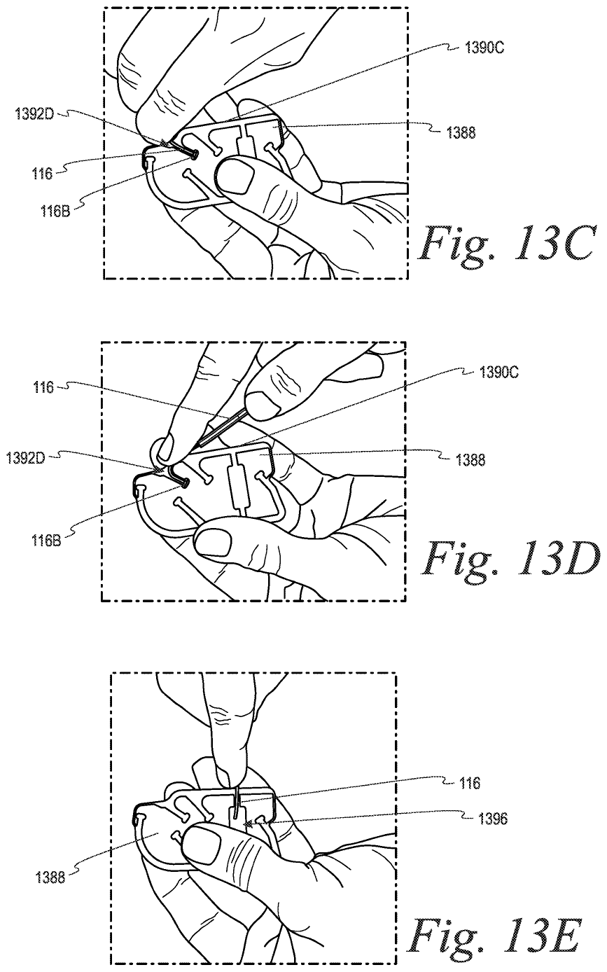

FIG. 10N depicts an arrangement for coupling the one or more signal leads 1064 to the wire coil 352, according to one example. As shown in FIG. 10N, the ferromagnetic core 350 can include a notch 1011 that extends distally from a proximal end 1050A of the ferromagnetic core 350. A distal end of each signal lead 1064 can be coupled to the wire coil 352 in a space defined by the notch 1011. In this arrangement, the ferromagnetic core 350 can help to mitigate bending at a location of the coupling between the signal lead(s) 1064 and the wire coil 352 and, therefore, help to mitigate damage to the coupling when bending the shaft 112 to form the curved segment 232 as described above and as described in further detail below with respect to FIGS. 13A-13E. For instance, as shown in FIG. 10N, the wire coil 352 can be wrapped around and soldered to the distal ends 1064A of the signal lead(s) 1064 in some examples. The ferromagnetic core 350 extends circumferentially around the shaft 112 on opposing sides of the coupling between the signal lead(s) 1064 and the wire coil 352, which can help to enhance structural rigidity of the shaft 112, resist bending at the coupling, and mitigate a risk of the solder being compromised. In examples in which the ferromagnetic core 350 include a coating of an insulator material, the coating can extend over the notch 1011 and the coupling between the signal lead(s) 1064 and the wire coil 352 in the notch 1011.

FIG. 10O depicts an arrangement for coupling the one or more signal leads 1064 to the wire coil 352, according to another example. As shown in FIG. 10O, in some examples, the signal lead(s) 1064 can be coupled to the wire coil 352 by a flex circuit board 1013. The flex circuit board 1013 can provide a more gradual transition in stiffness from the wire coil 352 to the signal lead(s) 1064 and/or provide for simplified manufacturing. For example, the wire coil 352 can have a first stiffness, the flex circuit board 1013 can have a second stiffness, and the signal lead(s) 1064 can have a third stiffness, where the first stiffness is less than the second stiffness and the second stiffness can be less than the third stiffness. The more gradual transition in stiffness can help to reduce a risk of decoupling due to bending as compared, for instance, to a direct coupling between the signal lead(s) 1064 and the wire coil 352. Additionally or alternatively, the flex circuit board 1013 can be configured to couple to the signal lead(s) 1064 and the wire coil 252 by a respective solder coupling (e.g., without the winding coupling described above and shown in FIG. 10N). This can help to simplify manufacture and/or assembly.

In some examples (e.g., the example shown in FIGS. 10E-10G), the position sensor 130 can extend to distal end 116B of the hypotube 116. This can help to directly sense a position of the distal end 116B of the hypotube 116 and/or the atraumatic tip 1040. In other examples, the position sensor 130 can be at a position that is offset in a proximal direction from the distal end 116B of the hypotube 116. This can help to mitigate the position sensor 130 contacting tissue. In some examples, the position sensor 130 can be at a position that is proximal of the atraumatic tip 1040 as well. This can help to reduce or minimize interference from the atraumatic tip 1040, which may be relatively thicker than a portion of the hypotube 116 that is proximal of the atraumatic tip 1040.

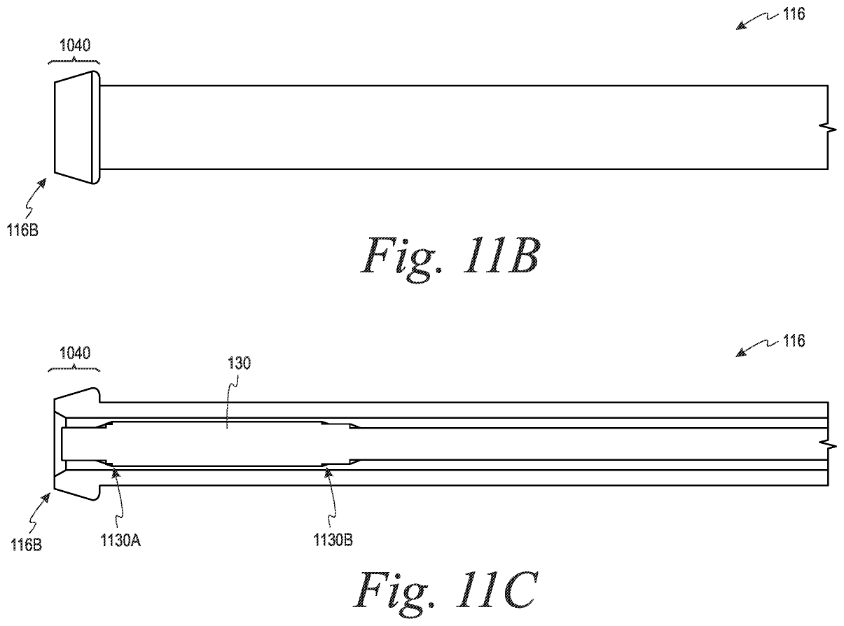

FIGS. 11A-11C depict an example implementation of the balloon dilation device 100 shown in FIGS. 10A-10M, except the position sensor 130 is proximal of the distal end 116B and/or the atraumatic tip 1040. FIG. 11A depicts a perspective view of the balloon dilation device 100, FIG. 11B depicts a distal portion of the hypotube, and FIG. 11C depicts a cross-section view of the distal portion of the hypotube 116 shown in FIG. 11B, according to the example implementation. The balloon dilation device shown in FIG. 11A is identical to the balloon dilation device 100 shown in FIGS. 10A-10M, except the position sensor 130 is at a location that is proximal of the distal end 116B of the hypotube 116 and the atraumatic tip 1040.

As shown in FIG. 11C, the position sensor 130 includes a distal sensor end 1130A and a proximal sensor end 1130B. A distalmost portion of the wire coil 352 shown in FIGS. 10F-10G is located at the distal sensor end 1130A in FIG. 11C, and a proximal-most portion of the wire coil 352 is located at the proximal sensor end 11030B in FIG. 11C. As shown in FIG. 11C, the distal sensor end 1130A is proximal of the distal end 116B of the hypotube 116 and the atraumatic tip 1040.

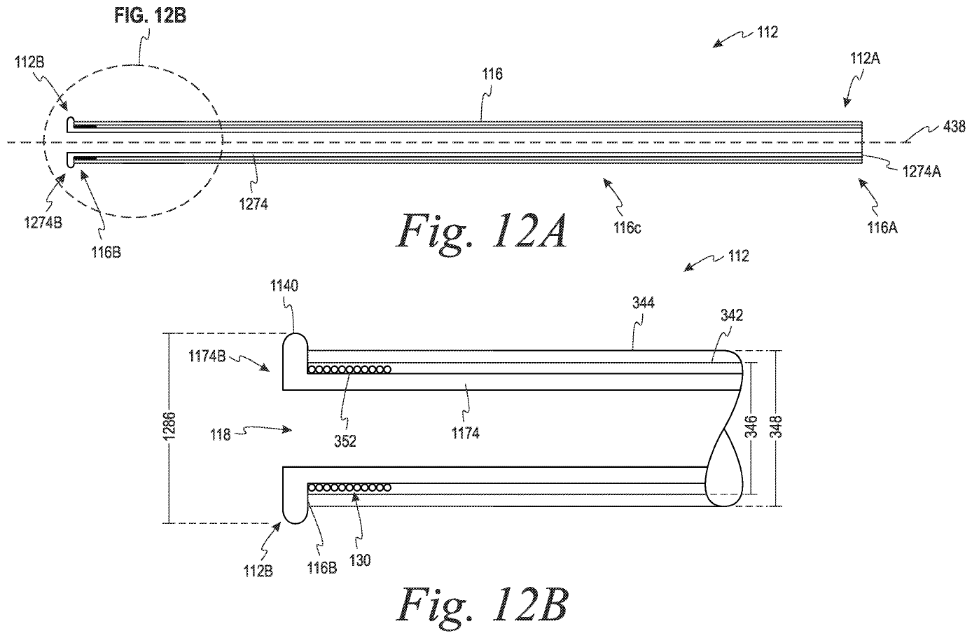

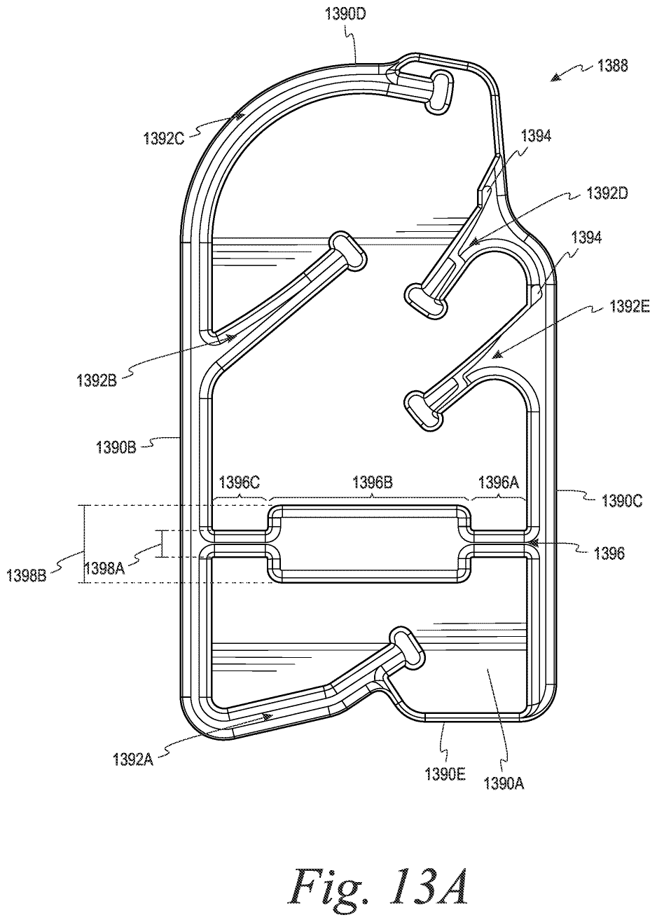

FIGS. 12A-12B depict another implementation for the shaft 112 that can be used with the balloon dilation device shown in FIGS. 10A-10D and 11, according to another example. FIG. 12A depicts a side view of the shaft 112, and FIG. 12B depicts a cross-sectional view of a portion of the shaft 112 shown in FIG. 12A taken through a longitudinal axis 438 of the shaft 112 according to the example. As shown in FIG. 12A, the shaft 112 extends between the proximal end 112A and the distal end 112B of the shaft 112.

As shown in FIGS. 12A-12B, the shaft 112 includes the hypotube 116 and the position sensor 130. The hypotube 116 has the proximal end 116A, the distal end 116B, and the body portion 116C between the proximal end 116A and the distal end 116B of the hypotube 116. In this example, the proximal end 116A of the hypotube 116 can correspond to the proximal end 112A of the shaft 112. As shown in FIG. 12B, the hypotube 116 also includes the inner surface 342 and the outer surface 344. The inner surface 342 defines the inner diameter 346 of the hypotube 116, and the outer surface 344 defines the outer diameter 348 of the hypotube 116. The inner surface 342 defines a lumen of the hypotube 116.

Additionally, as shown in FIGS. 12A-12B, the shaft 112 includes a liner tube 1274 disposed within the lumen of the hypotube 116. The shaft 112 includes the position sensor 130 coupled to a distal portion of the hypotube 116, and the position sensor 130 is between the inner surface 342 of the hypotube 116 and the liner tube 1274. For instance, as shown in FIG. 12B, the position sensor 130 can include the wire coil 352 disposed around the liner tube 1274 (and within the lumen of the hypotube 116). In this arrangement, the liner tube 1274 can define the working lumen 118 of the shaft 112.

As shown in FIG. 12A, the liner tube 1274 extends between a proximal end 1274A and a distal end 1274B. As shown in FIG. 12B, the distal end 1274B of the liner tube 1274 is distal of the distal end 116B of the hypotube 116. Additionally, as shown in FIG. 12B, the distal end 1274B of the liner tube 1274 can define an atraumatic tip 1140 of the shaft 112. In some examples, the liner tube 1274 and the atraumatic tip 1140 can be formed from a polymer material. This can help to provide a cushion between the distal end 116B of the hypotube 116, which can be made of a substantially rigid material, and the anatomy of the patient. As described above, the atraumatic tip 1140 can have a bulbous shape and/or a bullet shape. For instance, as shown in FIGS. 12A-12B, a diameter 1286 of the atraumatic tip 1140 can be greater than the outer diameter 348 of the outer surface 344 of the hypotube 116 at the distal end 116B of the hypotube 116.

As shown in FIG. 12A, the proximal end 1274A of the liner tube 1274 can be at the proximal end 116A of the hypotube 116. In some examples, the liner tube 1274 can be single, monolithic structure with the distal end 1274B of the liner tube 1274 extending distally of the distal end 116B of the hypotube 116 and the proximal end 1274A of the liner tube 1274 at a proximal end 116A of the hypotube 116. This can help to provide a continuous surface within the working lumen 118, which can help to mitigate a light fiber snagging as the light fiber 1072 is inserted and/or retracted through the working lumen 118. Although it can be beneficial to have liner tube 1274 continuously extend along at least the entire length of the lumen of the hypotube 116, the liner tube 1274 can be formed by a plurality of separate components coupled together and/or separated by gaps in other examples.

Although not shown in FIGS. 12A-12B, the position sensor 130 can also include the ferromagnetic core 350 between the liner tube 1274 and the wire coil 352, for instance, in the arrangement shown in FIGS. 10F-10G. As described above, this can help to reduce a quantity of coils of the wire coil 352 and/or enhance a quality of the signal generated by the position sensor 130 responsive to the electromagnetic field at the surgical site.

Additionally, although not shown in FIGS. 12A-12B, the shaft 112 can additionally or alternatively include the protective cover 356 that covers the wire coil 352 between the wire coil 352 and the inner surface 342 of the hypotube 116 (e.g., as shown in FIGS. 10F-10G). As described above, the protective cover 356 can help to retain and protect the wire coil 352 (e.g., help mitigate scratching the wire coil 352 and/or the hypotube 116). The protective cover 356 can additionally or alternatively help to electrically insulate the wire coil 352 from the hypotube 116.

Additionally, as described above with respect to FIGS. 10A-10D, the balloon dilation device 100 can include one or more signal leads (e.g., the one or more signal leads 1064 shown in FIG. 10F) disposed between the liner tube 1074 and the inner surface 342 of the hypotube 116, extending through the working lumen 118 (e.g., inside of the liner tube 1274) and/or embedded in the liner tube 1274.

As described above, in some examples, the distal portion of the shaft 112 can be malleable, and the user can form the curved segment 232 of the shaft 112 by bending the distal portion of the shaft 112 prior to inserting the balloon dilation device 100 into the nasal cavity of the patient. FIG. 13A depicts a bending tool 1388 that is configured to form the curved segment 232 of the shaft 112 with one or more degrees of curvature. The bending tool 1388 can include a front surface 1390A (shown in FIG. 13A), a first lateral side 1390B, a second lateral side 1390C, an upper side 1390D, a lower side 1390E, and a back side (not shown). The bending tool 1388 can be formed from a rigid material such as, for instance, a thermoplastic material.

As shown in FIG. 13A, the bending tool 1388 can include a plurality of bend channels 1392A-1392E that are configured to bend the hypotube 116 at a plurality of different bend angles to form the curved segment 232 of the shaft 112. In this example, each bend channel 1392A-1392E is configured to form a respective bend angle that is different than the other bend angles. For instance, the bend channels 1392A-1392E can include (i) a first bend channel 1392A that is configured to form a bend angle in the shaft 112 that is suitable for accessing the sphenoid sinus (e.g., a bend angle of approximately 16 degrees plus or minus 5 degrees), (ii) a second bend channel 1392B that is configured to form a bend angle in the shaft 112 that is suitable for accessing the Eustachian tube (e.g., a bend angle of approximately 45 degrees), (iii) a third bend channel 1392C that is configured to form a bend angle in the shaft 112 that is suitable for accessing the frontal sinus (e.g., a bend angle of approximately 80 degrees plus or minus 10 degrees), (iv) a fourth bend channel 1392D that is configured to form a first bend angle in the shaft 112 that is suitable for accessing the maxillary sinus (e.g., a bend angle of approximately 135 degrees), and (v) a fifth bend channel 1392E that is configured to form a second bend angle in the shaft 112 that is suitable for accessing the maxillary sinus (e.g., a bend angle of approximately 120 degrees).

In use, the distal end 116B of the hypotube 116 can be inserted into one of the bend channels 1392A-1392E such that the distal end 116B is at the end of the channel 1392A-1392E and a first portion of the hypotube 116 extends in the channel 1392A-1392E. While the atraumatic tip 1040 is at the end of the channel 1392A-1392E and the first portion of hypotube 116 is in the channel 1392A-1392E, a second portion of the shaft 112 that is external to the channel 1392A-1392E can be manually bent around curved surfaces at the first lateral side 1390B and the second lateral side 1390C (e.g., the second portion of the shaft 112 can be manually bent towards the first lateral side 1390B for the channels 1392A-1392C or the second lateral side 1390C for the channels 1392D-1392E) to form the curved segment 232 with the bend angle that corresponds to the channel 1392A-1392E in which the hypotube 116 was inserted. As an example, FIG. 13C depicts the distal end 116B and the first portion of the hypotube 116 inserted into the fourth bend channel 1392D prior to forming a bend using the bending tool 1388, and FIG. 13D depicts the hypotube 116 being manually bent towards the second lateral side 1390C to form a bend in the hypotube 116.

In some instances, it can be desirable to unbend the shaft 112. For example, after performing a procedure on one sinus, a user can use the balloon dilation device 100 on another sinus. To facilitate accessing the next sinus to be treated, the user can use the bending tool 1388 to change the bend angle at the curved segment 232 of the shaft 112. In some implementations, a process for changing the bend angle at the curved segment 232 of the shaft 112 can include unbending the shaft 112 and, after unbending the shaft 112, using the bending tool 1388 to form the curved segment 232 with a different bend angle (e.g., by using a different one of the channels 1390A-1390E than was used previously).

Within examples, the bending tool 1388 can be used to unbend the shaft 112. For instance, the channels 1392A-1392E can be used to at least partially unbend the shaft 112. To unbend the shaft 112, the distal end 116B of the hypotube 116 can be inserted into one of the bend channels 1392A-1392E such that the distal end 116B is at the end of the channel 1392A-1392E and a first portion of the hypotube 116 extends in the channel 1392A-1392E. While the atraumatic tip 1040 is at the end of the channel 1392A-1392E and the first portion of hypotube 116 is in the channel 1392A-1392E, the second portion of the shaft 112 that is external to the channel 1392A-1392E can be manually bent in a direction that is opposite a direction of the bend at the curved segment 232 of the shaft 112 (e.g., a direction opposite to the curved surfaces at the first lateral side 1390B and the second lateral side 1390C). For example, the distal end 116B and the first portion of the hypotube 116 can be inserted into the fourth bend channel 1392D as shown in FIG. 13D, and manually bent away from the second lateral side 1390C to the position shown in FIG. 13C to unbend the hypotube 116.

To help mitigate the shaft 112 inadvertently decoupling from the bend channel 1392A-1392E while unbending the shaft 112 (e.g., by popping out through the front surface 1390A), the bending tool 1388 can include one or more undercuts 1394 on the front surface 1390A and along at least a portion of the channel 1392A-1392E. As an example, FIG. 13B depicts the hypotube 116 in the fourth bend channel 1392D and a plurality of undercuts 1394 that can help to retain the hypotube 116 in the fourth bend channel 1392D while unbending the shaft 112. As shown in FIG. 13B, each undercut 1394 can extend laterally from a side of the fourth bend channel 1394D over the hypotube 116. However, the undercuts 1394 do not extend entirely across the fourth bend channel 1394D. Rather, as shown in FIG. 13B, the undercuts 1394 provide a gap that allows the hypotube 116 to be inserted into the fourth bend channel 1394D from above and in a direction from the front surface 1390A towards the back surface.

In FIG. 13A, the bending tool 1388 includes the undercuts 1394 at the fourth bend channel 1392D and the fifth bend channel 1392E. This can be beneficial as it may be particularly challenging to unbend the shaft 112 when the curved segment 232 has a bend that was previously formed by the fourth bend channel 1392D or the fifth bend channel 1392E for accessing the maxillary sinus. In other examples, the bending tool 1388 can additionally or alternatively include the undercuts 1394 at the first bend channel 1392A, the second bend channel 1392B, and/or the third bend channel 1394C.

In some implementations, it may be challenging to fully unbend the shaft 112 using the bend channels 1392A-1392E and/or manually bending the shaft 112 with only the fingers of the user. To help unbend the shaft 112 more completely and efficiently, the bending tool 1388 can include a straightening channel 1396 that is configured to unbend the shaft 112. The straightening channel 1396 can include a first section 1396A having a first height 1398A and a second section 1396B having a second height 1398B, and the second height 1398B can be greater than the first height 1398A. The first height 1398A and the second height 1398B can be dimensions that extend between the upper side 1390D and the lower side 1390E. The first section 1396A can be between the second section 1396B and one of the first lateral side 1390B or the second lateral side 1390C.

In this arrangement, the distal end 116B of the hypotube 116 can be positioned in the second section 1396B of the straightening channel 1398, a first portion of the hypotube 116 can extend through the first section 1396A, and a second portion of the hypotube 116 can be external to the straightening channel 1398. The second height 1398B being greater than the first height 1398A can allow the second section 1396B to accommodate the distal end 116B of the hypotube 116 while the hypotube 116 is bent at least to some extent at the curved segment 232. As shown in FIG. 13E, after positioning the hypotube 116 in the straightening channel 1396, the second portion of hypotube 116 can be manipulated against walls of the first section 1396A of the straightening channel 1396 to straighten the hypotube 116 (e.g., the second portion of the hypotube 116 can be moved in an upper direction towards the upper side 1390D and/or a lower direction towards the lower side 1390E to straighten the hypotube 116). Additionally, the hypotube 116 can be moved in a lateral direction towards the first lateral side 1390B and/or the second lateral side 1390C to progressively move the hypotube 116 more distal portions of the hypotube 116 into the first section 1396A of the straightening channel 1396. In this way, the straightening channel 1396 can provide for straightening the hypotube 116 in a progressive manner in a direction from a proximal portion of the hypotube 116 towards a distal portion of the hypotube 116.

As shown in FIG. 13A, the first section 1396A can have a first width, the second section 1396B can have a second width, and the second width can be greater than the first width. The first width and the second width can extend in a dimension between the first lateral side 1390B and the second lateral side 1390C. The second width of the second section 1396B being larger than the first width of the first section 1396A can help provide greater space for receiving the distal end 116B of the hypotube 116 when the hypotube 116 includes a bend.

As shown in FIG. 13A, in some examples, the straightening channel 1396 can further include a third section 1396C, and the first section 1396A and the third section 1396C can be on opposite sides of the second section 1396B. As shown in FIG. 13A, the third section 1396C can have a similar or identical configuration to the first section 1396A (e.g., the third section 1396C can have the first height 1398A and the first length as described above with respect to the first section 1396A). In this example, the first section 1396A between the second lateral side 1390C and the second section 1396B, the second section 1396B extends between the first section 1396A and the third section 1396C, and the third section 1396C extends between the first lateral side 1390B and the second section 1396B. In this arrangement, the straightening channel 1396 can provide for right-handed and left-handed operation of the bending tool 1388 to unbend the hypotube 116.

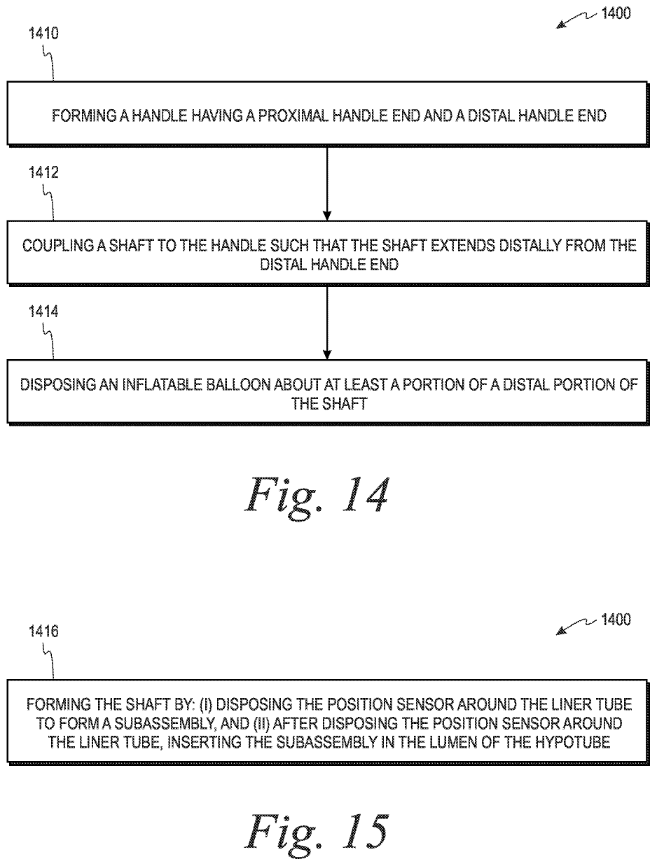

Referring to FIG. 14, a flowchart for a process 1400 of forming a balloon dilation device is shown according to an example. As shown in FIG. 14, the process 1400 includes forming a handle having a proximal handle end and a distal handle end at block 1410. The process 1400 also includes coupling a shaft to the handle such that the shaft extends distally from the distal handle end at block 1412. The shaft includes a hypotube having an inner surface and an outer surface. The inner surface defines a lumen of the hypotube. The shaft also includes a liner tube disposed within the lumen of the hypotube, and a position sensor coupled to a distal portion of the hypotube. The position sensor is between the inner surface of the hypotube and the liner tube. The process 1400 further includes disposing an inflatable balloon about at least a portion of a distal portion of the shaft at block 1414.

FIG. 15 depict additional aspects of the process 1400 according to further examples. As shown in FIG. 15, the process 1400 can also include forming the shaft by: (i) disposing the position sensor around the liner tube to form a subassembly, and (ii) after disposing the position sensor around the liner tube, inserting the subassembly in the lumen of the hypotube at block 1416.

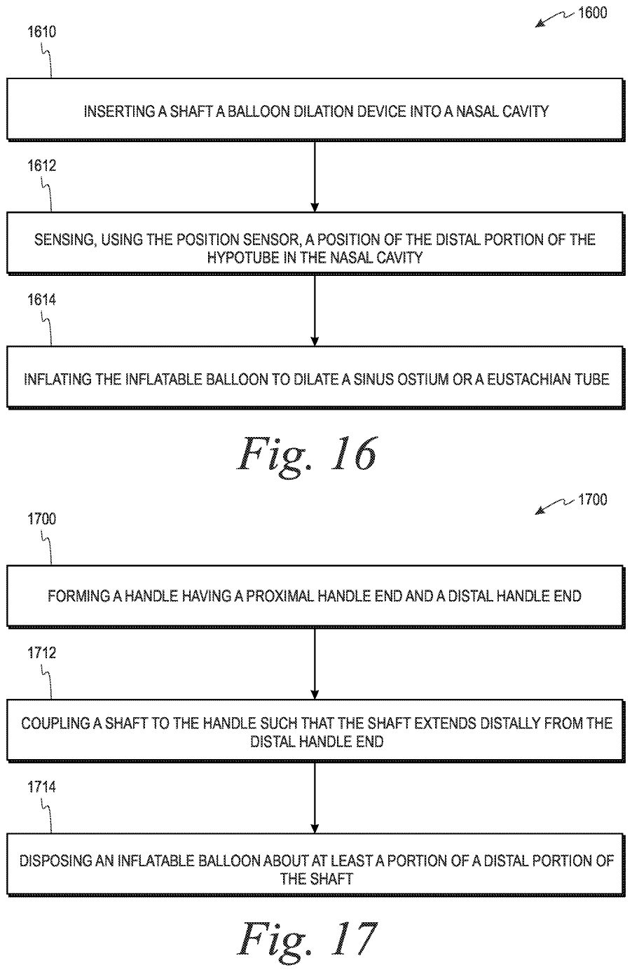

Referring to FIG. 16, a flowchart for a process 1600 of using a balloon dilation device is shown according to an example. At block 1610, the process 1600 includes inserting a shaft a balloon dilation device into a nasal cavity. The balloon dilation device includes a handle having a proximal handle end and a distal handle end, the shaft extending distally from the distal handle end, and an inflatable balloon disposed about at least a portion of a distal portion of the shaft. The shaft includes a hypotube including an inner surface and an outer surface. The inner surface defines a lumen of the hypotube. The shaft also includes a liner tube disposed within the lumen of the hypotube, and a position sensor coupled to a distal portion of the hypotube. The position sensor is between the inner surface of the hypotube and the liner tube. At block 1612, the process 1600 also includes sensing, using the position sensor, a position of the distal portion of the hypotube in the nasal cavity. At block 1614, the process 1600 includes inflating the inflatable balloon to dilate a sinus ostium or a Eustachian tube.

Referring to FIG. 17, a flowchart for a process 1700 of forming a balloon dilation device is shown according to an example. At block 1710, the process 1700 includes forming a handle having a proximal handle end and a distal handle end. At block 1712, the process 1700 includes coupling a shaft to the handle such that the shaft extends distally from the distal handle end. The shaft includes a hypotube having a proximal end, a distal end, and a body portion between the proximal end and the distal end of the hypotube. The hypotube includes an inner surface and an outer surface. The inner surface defines an inner diameter of the hypotube. The outer surface defines an outer diameter of the hypotube. The shaft also includes a position sensor coupled to a distal portion of the hypotube. At least a portion of the position sensor is in a volume of space defined by (i) an inner radius that is equal to the inner diameter of the hypotube at the distal portion of the hypotube and (ii) an outer radius that is equal to the outer diameter of the hypotube at the distal portion of the hypotube. At block 1714, the process 1700 includes disposing an inflatable balloon about at least a portion of a distal portion of the shaft.

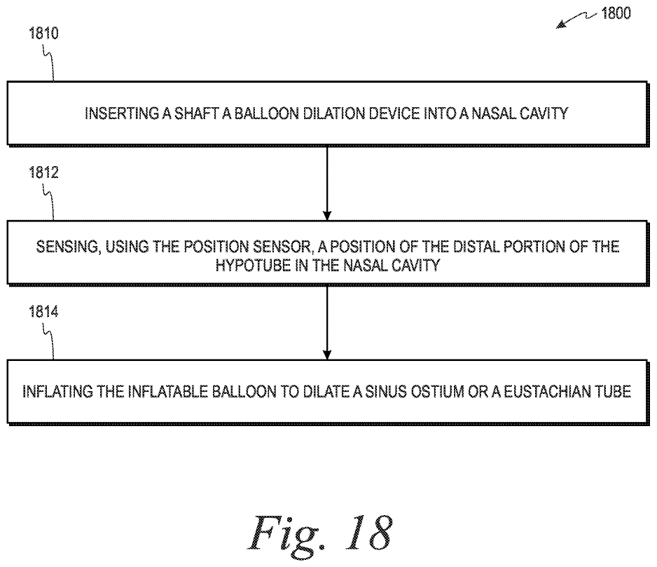

Referring to FIG. 18, a flowchart for a process 1800 of using a balloon dilation device is shown according to an example. At block 1810, the process 1800 includes inserting a shaft a balloon dilation device into a nasal cavity. The balloon dilation device includes a handle having a proximal handle end and a distal handle end, a shaft extending distally from the distal handle end, and an inflatable balloon disposed about at least a portion of a distal portion of the shaft.

The shaft includes a hypotube having a proximal end, a distal end, and a body portion between the proximal end and the distal end of the hypotube. The hypotube includes an inner surface and an outer surface. The inner surface defines an inner diameter of the hypotube. The outer surface defines an outer diameter of the hypotube. The shaft also includes a position sensor coupled to a distal portion of the hypotube. At least a portion of the position sensor is in a volume of space defined by (i) an inner radius that is equal to the inner diameter of the hypotube at the distal portion of the hypotube and (ii) an outer radius that is equal to the outer diameter of the hypotube at the distal portion of the hypotube.

At block 1812, the process 1800 includes sensing, using the position sensor, a position of the distal portion of the hypotube in the nasal cavity. At block 1814, the process 1800 includes inflating the inflatable balloon to dilate a sinus ostium or a Eustachian tube.

The description of the different advantageous arrangements has been presented for purposes of illustration and description, and is not intended to be exhaustive or limited to the examples in the form disclosed. Many modifications and variations will be apparent to those of ordinary skill in the art. Further, different advantageous examples may describe different advantages as compared to other advantageous examples. The example or examples selected are chosen and described in order to explain the principles of the examples, the practical application, and to enable others of ordinary skill in the art to understand the disclosure for various examples with various modifications as are suited to the particular use contemplated.

Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present application is not to be limited by the subject specification, but rather only by the plain meaning of the claim terms employed.

1. A balloon dilation device, comprising:
a handle having a proximal handle end and a distal handle end;
a shaft extending distally from the distal handle end, wherein the shaft comprises:
a hypotube comprising an inner surface and an outer surface, wherein the inner surface defines a lumen of the hypotube,
a liner tube disposed within the lumen of the hypotube, and
a position sensor coupled to a distal portion of the hypotube, wherein the position sensor is between the inner surface of the hypotube and the liner tube; and
an inflatable balloon disposed about at least a portion of a distal portion of the shaft.

2. The balloon dilation device of claim 1, wherein the position sensor comprises a wire coil disposed around the liner tube.

3. The balloon dilation device of claim 2, wherein the position sensor further comprises a ferromagnetic core between the liner tube and the wire coil.

4. The balloon dilation device of claim 2, wherein the shaft further comprises a protective cover that covers the wire coil between the wire coil and the inner surface of the hypotube.

5. The balloon dilation device of claim 4, wherein a proximal end of the protective cover is proximal of a proximal end of the wire coil.

6. The balloon dilation device of claim 2, further comprising one or more signal leads that extend from the wire coil to a proximal end of the shaft, wherein the one or more signal leads are disposed between the liner tube and the inner surface of the hypotube.

7. (canceled)

8. The balloon dilation device of claim 1, wherein the liner tube defines a working lumen that extends from a distal end of the shaft to a proximal end of the shaft, and wherein the balloon dilation device further comprises a light fiber that is movable in the working lumen.

9. (canceled)

10. The balloon dilation device of claim 8, further comprising a light source and a power source in an internal cavity defined by the handle.

11. The balloon dilation device of claim 10, wherein a slack portion of the light fiber extends out of the handle, wherein the slack portion is configured to be inserted into the handle and retracted out of the handle to extend and retract, respectively, a distal portion of the light fiber from a distal end of the shaft.

12. The balloon dilation device of claim 8, wherein the light fiber and the working lumen have respective diameters such that an air gap is formed between the light fiber and the working lumen, wherein the air gap defines an airflow pathway between the distal end of the shaft and a vent opening in the handle.

13-65. (canceled)

66. A balloon dilation device, comprising:
a handle having a proximal handle end and a distal handle end;
a shaft extending distally from the distal handle end, wherein the shaft comprises:
a hypotube comprising an inner surface and an outer surface, wherein the inner surface defines a lumen of the hypotube,
a liner tube disposed within the lumen of the hypotube, wherein a distal end of the liner tube is distal of a distal end of the hypotube and defines an atraumatic tip of the shaft, and
a position sensor coupled to a distal portion of the hypotube, wherein the position sensor is between the inner surface of the hypotube and the liner tube; and
an inflatable balloon disposed about at least a portion of a distal portion of the shaft.

67. The balloon dilation device of claim 66, wherein the liner tube is formed from a polymer material.

68. (canceled)

69. The balloon dilation device of claim 66, wherein a proximal end of the liner tube is at a proximal end of the hypotube.

70. The balloon dilation device of claim 66, wherein the liner tube is a single, monolithic structure between the distal end and the proximal end of the liner tube.

71. The balloon dilation device of claim 66, wherein the position sensor comprises a wire coil disposed around the liner tube.

72. The balloon dilation device of claim 71, wherein the position sensor further comprises a ferromagnetic core between the liner tube and the wire coil.

73. The balloon dilation device of claim 71, wherein the shaft further comprises a protective cover that covers the wire coil between the wire coil and the inner surface of the hypotube.

74. The balloon dilation device of claim 72, wherein the ferromagnetic core comprises an electrically insulative coating on an exterior surface of the ferromagnetic core that faces the wire coil to help mitigate shorting between the ferromagnetic core and the wire coil.

75. The balloon dilation device of claim 72, wherein the position sensor further comprises one or more signal leads that extend from the wire coil to a proximal end of the of the shaft,
wherein the ferromagnetic core comprises a notch that extends distally from a proximal end of the ferromagnetic core, and
wherein a distal end of each signal lead is coupled to the wire coil in a space defined by the notch.

76. The balloon dilation device of claim 72, wherein the position sensor further comprises one or more signal leads that extend from the wire coil to a proximal end of the of the shaft,
wherein the one or more signal lead(s) are coupled to the wire coil by a flex circuit board,
wherein the wire coil has a first stiffness, the flex circuit board has a second stiffness, and the one or more signal leads have a third stiffness, and
wherein the first stiffness is less than the second stiffness and the second stiffness is less than the third stiffness.

77. The balloon dilation device of claim 66, wherein the position sensor is at a position that is offset in a proximal direction from a distal end of the hypotube.
